(19) United States
(12) Patent Application Publication
(10) Pub. No.: US 2008/0227752 A1
(43) Pub. Date: Sep. 18, 2008

(54) Methods to Inhibit Histone Acetyltransferase Using Glycosaminoglycans

(75) Inventors: Matthew A. Nugent, Bedford, MA (US); Edward Hsia, Foster City, CA (US); Jo Ann Buczek-Thomas, Easton, MA (US)

Correspondence Address: RONALD I. EISENSTEIN, 100 SUMMER STREET, NIXON PEABODY LLP, BOSTON, MA, 02110, US

(73) Assignee: Trustees of Boston University, Boston, MA (US)

(21) Appl. No.: 11/630,078

(22) PCT Filed: Jun. 28, 2005
(86) PCT No.: PCT/US2005/022905

§ 371 Date: Oct. 30, 2007

Related U.S. Application Data
(60) Provisional application No. 60/584,358, filed on Jun. 30, 2004.

(51) Int. Cl. A61K 31/715; A61P 27/02; A61P 11/00; A61P 35/04; A61P 9/00

(52) U.S. Cl. 514/56; 514/54

(57) ABSTRACT

The present invention is directed to methods for inhibition of histone acetyltransferases using glycosaminoglycans. The invention is further directed to methods for treating disorders associated with hyperacetylation by administration of glycosaminoglycans to a patient in need thereof. In one preferred embodiment, the glycosaminoglycan is a heparin or heparan sulfate oligosaccharide. Studies show that removal of sulfate residues from the O-positions of either the uronic acid or the glucosamine did not eliminate the inhibitory activity of heparan sulfate. Since a majority of heparan sulfate binding proteins appear to require O-sulfation, molecules without certain O-sulfations can be used to inhibit HATs while not interacting with most known heparin-binding proteins. In addition, specific sequences of heparin/heparan sulfate can be used to specifically inhibit various HATs.

### CROSS-REFERENCE TO RELATED APPLICATIONS

The present application claims the benefit under 35 U.S.C. 119 (e) of the U.S. provisional Patent Application No. 60/584,358, filed Jun. 30, 2004.

### GOVERNMENT SUPPORT

This invention was made with Government support under contract Nos. EY14007 and HL46902 awarded by the National Institutes of Health. The Government of the United States has certain rights to the invention.

### FIELD OF THE INVENTION

The present application relates to the use of glycosaminoglycans (e.g. heparin, heparan sulfate, chrondroitin sulfate, keratan sulfate, and hyaluronan) as inhibitors of histone acetyltransferase (HATs) activity and to their use for treatment of disorders associated with hyperacetylation.

### BACKGROUND OF THE INVENTION

Histone acetyltransferase (HAT) complexes are involved in diverse processes such as transcription activation, gene silencing, DNA repair and cell-cycle progression. The high evolutionary conservation of the acetyltransferase complexes and their functions also illustrates their central role in cell growth and development.

Modification of histone tails by acetylation is known to increase the access of transcription factors to DNA through structural changes in chromatin structure (e.g. nucleosomes or nucleosomal arrays) (Brown et al. 2000; Sterner and Berger 2000; Gregory et al. 2001; Marmorstein and Roth 2001). The structural changes create and/or eliminate binding sites for transcription factors. For example, CREB-binding protein (p300/CBP), which has a histone acetyltransferase domain has been shown to be a co-activator of transcription factor p53 by increasing its DNA-binding capacity, enhancing its stability, and effecting its interaction with other proteins (Gu and Roeder 1997; Luo et al. 2000; Li et al. 2002; Brooks and Gu 2003).

The first transcription-related HAT was discovered in 1996 (Brownell et al. 1996). Since then, over 25 members falling into five distinct families have been discovered in organisms spanning from yeast to humans. In addition to the relationship between histone acetylation and the transcriptional capacity of chromatin, acetylation by HATs is also involved in processes such as replication and nucleosome assembly (Grant, P A & Berger, S L 1999). HATs are further believed to acetylate other HATs and act as signal transducers similar to kinases in phosphorylation cascades (Kouzarides 2000).

Hyperacetylation within cells mediated by histone acetyltransferases is associated with a hypoproliferative phenotype and leads to a variety of disorders such as cancer, cardiovascular disease, proliferative eye disease, psoriasis, diabetic retinopathy, arthritis and chronic obstructive pulmonary disease, as well as others. Cigarette smoking has been linked to the development of chronic obstructive pulmonary disease and cigarette smoke has been shown to increase histone 4 acetylation (Marwick, J. A., et al., 2004; Rahman, I., et al. 2004). Recently, in humans, increased histone acetylation has been associated with emphysema as the result of insufficient histone deacetylase activity (Ito, K., et al. 2005). Moreover, corticosteroid resistance in chronic obstructive pulmonary disease has been attributed to inactivation of histone deacetylase which can be restored by treatment with theophylline (Cosio, B. G., et al. 2004; Barnes, P. J. 2003; Barnes, P. J., et al., 2004).

Accordingly, there have been efforts to identify inhibitors of histone acetyltransferases (See U.S. Pat. Nos. 6,369,030 and 6,747,005). Pharmacological agents have been developed that aim to modulate HAT activity, particularly as a treatment for various forms of cancer (U.S. Patent Application 20040091967). However, these inhibitors are not highly specific and often have undesirable side effects.

Glycosaminoglycans, known to have roles in inflammation, proliferation, and/or anti-coagulant effects have been reported to be involved in treating a number of disorders (U.S. Patent Publication No. 20020086852; 20030086899; U.S. Pat. Nos. 6,159,954; 5,795,875;6,537,978 and 5,980,865).

There is a need in the art to identify specific inhibitors of HAT activity that are safe, effective and specific, so that disorders associated with hyperacetylation can be effectively treated.

### SUMMARY OF THE INVENTION

The present application is based on the discovery that glycosaminoglycans are potent inhibitors of histone acetyltransferases (HATs). For example, the oligosaccharides heparin, heparan sulfate, chrondroitin sulfate, keratan sulfate, and hyaluronic acid inhibit HATs; heparin and heparan sulfates are potent inhibitors of HAT (e.g. p300 and pCAF HAT). Further, the unique structures of oligosaccharides provide a means for specific inhibition of histone acetyltransferases in treatment of disorders associated with excessive HAT activity.

In one embodiment, a method is provided for inhibiting a histone acetyltransferase. The method comprises contacting histone acetyltransferase, or a substrate of a histone acetyltransferase, with a glycosaminoglycan, e.g. heparin, heparan sulfate oligosaccharide, heparan sulfate proteoglycan (HSPG), chrondroitin sulfate, keratan sulfate and hyaluronic. Preferably the inhibitor is HS or HSPG. Preferably, the heparan sulfate, or HSPG contains N-sulfation.

In another embodiment, a method is provided for treating a disorder associated with hyperacetylation comprising administration of an effective amount of a pharmaceutical composition containing as its active agent a glycosaminoglycan oligosaccharide to a patient having the disorder, wherein an effective amount is an amount sufficient to inhibit a histone acetyltransferase. The active agent glycosaminoglycan includes heparin or heparan sulfate oligosaccharide, hyaluronan, chrondroitin sulfate, or keratan sulfate; or derivatives thereof.

Preferably, the glycosaminoglycans of the invention are oligosaccharides of at least 5 or 6 sugars in length. More preferably, 8-18 sugars in length. Even more preferably, 8-12 sugars in length.

In one embodiment, the glycosaminoglycan used as the active agent is chemically or enzymatically modified as to alter their pattern of sulfation.

In one embodiment, the pharmaceutical composition containing as its active agent a glycosaminoglycan oligosaccharide further comprises an agent that enhances nuclear uptake of the glycosaminoglycan (e.g. a polyaminoester).

In one preferred embodiment, the active agent is heparin or heparan sulfate oligosaccharide. The active agent can be a heparin or heparan sulfate oligosaccharide or can be heparan sulfate proteoglycan ectodomain. Preferably the heparan sulfate proteoglycan ectodomain is isolated from corneal stromal fibroblasts or pulmonary fibroblasts.

In one preferred embodiment, the heparin or heparan sulfate oligosaccharide does not contain O-sulfation on the 2 position of the uronic acid residues.

In one preferred embodiment, the heparin or heparan sulfate oligosaccharides do not contain O-sulfation on the 6 position of glucosamine residues or on the 2 position of the uronic acid residues. Most preferably, the heparin or heparan sulfate oligosaccharides contain O-sulfation either on the 6 position of glucosamine residues, or on the 2 position of the uronic acid residues.

Any disorder associated with hyperacetylation can be treated by methods of the invention, for example cancer, proliferative eye disease, psoriasis, arthritis and chronic obstructive pulmonary disease, and cardiovascular disease.

In one preferred embodiment, the disorder to be treated is chronic obstructive pulmonary disease (e.g. asthma, bronchitis, and emphysema).

### BRIEF DESCRIPTION OF THE DRAWINGS

FIG. 1 shows a schematic representation of the domain structure of heparan sulfate (HS) attached to a transmembrane core protein to form a cell surface HS proteoglycan (HSPG). HSPG contain one or more covalently attached HS chains. These chains consist of unmodified regions, which are mostly N-acetylated and contain little sulfate, regions with a high level of epimerization and sulfation (S-domains), and regions with alternating N-acetylation and N-sulfation (NA/S-domains). This diagram depicts the most common structural modifications for these regions, but other minor modifications may also occur (R=OH or OSO₃) (Tumova et al. 2000).

FIG. 2 shows a schematic representation of one of the In Vitro HAT Activity Assays. Biotinylated histone H4 peptide (substrate) is incubated with recombinant HAT enzyme (p300 catalytic domain) and ³H-Acetyl-CoA, resulting in the covalent transfer of ³H-acetate to the substrate. The ³H-acetylated substrate can be extracted using immobilized streptavidin and counted in a scintillation counter. Thus, the amount of ³H-acetylated substrate is a direct measure of HAT enzyme activity. Additional assays for HAT activity use core histones as the substrate and the ³H-acetylated histone reaction products were separated from ³H-Acetyl-CoA by membrane filtration.

FIG. 3 is a graph indicating that heparin inhibits HAT Activity In Vitro. Various concentrations of heparin were incubated in the presence of biotinylated histone H4 peptide, recombinant p300 catalytic domain, and [³H]-acetyl-CoA for 1 hr at 30° C. Immobilized streptavidin was used to sequester the biotinylated substrate and was counted in a scintillation counter. Data presented are means of duplicates ±1 SE and are representative of five separate experiments. The amount of [³H] associated with the immobilized streptavidin-biotinylated substrate in the absence of HAT enzyme (negative control) was 54.2±12.0.

FIG. 4 shows a graph indicating that heparin binds biotinylated histone H4. Biotinylated histone H4 was incubated with sepharose [□], heparin-sepharose [Δ], or buffer only [⋄], in the presence of various concentrations of NaCl for 1 hr at room temp. Following centrifugation at 10,000×g for 10 min, the resulting supernatant was assayed for protein content. Values are expressed in percent relative to control (protein recovered in the supernatant in the absence of sepharose or heparin-separose):

$$( \frac{\begin{matrix}Protein recovered after incubation \\ with heparin \text{-} sepharose or sepharose\end{matrix}}{\begin{matrix}Protein recovered in the absence of \\ heparin \text{-} sepharose or sepharose\end{matrix}} ) \times 100$$

Data presented are means of duplicates and are representative of two separate experiments.

FIG. 5 shows a graph indicating that heparin binds recombinant HAT enzyme In Vitro. Recombinant p300 catalytic domain (0.04 U/mL) was incubated with a 1:1 slurry of sepharose or heparin-sepharose in the presence of increasing concentrations of NaCl for 1 h at room temp. Following centrifugation at 10,000×g for 10 min, the resulting supernatant was assayed for HAT activity. HAT activity is expressed in percent relative to sepharose: Data presented are means of duplicates ±SE and are representative of two separate experiments.

FIG. 6 shows that cell surface HSPG ectodomains (HSPGf) isolated from corneal stromal fibroblasts (CSF) inhibit HAT activity In Vitro. HAT activity was assessed in the presence of increasing concentrations of HSPGf. HSPGf was released from CSF by mild trypsin treatment and purified by Q-sepharose column chromatography. Relative concentration of glycosaminoglycans was determined by DMB assay using chondroitin sulfate as a standard (Farndale et al. 1986). Data presented are means of duplicates ±SE and are representative of four separate experiments. The negative control was 10.3±15.2 CPM. Estimated IC50 was 3 μg/mL.

FIGS. 7A and 7B shows that CABC-digested cell-surface HSPG fragments (HSPGf) are more potent than non-CABC-digested HSPGf in inhibiting HAT activity In Vitro. FIG. 7A, HAT activity was assessed in the presence of increasing concentrations of HSPGf that were previously digested with 0.005 U/mL chondroitinase ABC (CABC). CABC-digested HSPGf were isolated using Q-sepharose. Relative concentration of glycosaminoglycans was determined by DMB assay using chondroitin sulfate as a standard (Farndale et al. 1986). Data presented are means of duplicates ±SE and are representative of two separate experiments. The negative control was 18.5±1.1 CPM. Estimated IC50 was 1 μg/mL. FIG. 7B, comparison of dose response curves for heparin [∘], HSPGf [□], and CABC-digested HSPGf [Δ]. HAT activity is expressed as % of positive control.

FIG. 8 shows selective lyase digestion of HSPGf impacts HAT inhibition In Vitro. HAT activity was assessed in the presence HSPGf (2 μg/mL) that were previously digested with 0.005 U/mL chondroitinase ABC (CABC), 2 μg/mL heparinase I (Hep I), 0.1 U/mL heparinase III (Hep III), or not at all (HSPGf). Glycosaminoglycan concentrations were determined by DMB assay, using chondroitin sulfate as a standard. Data is presented as % Inhibition of HAT activity ±SE. Similar results were observed in two separate experiments.

FIG. 9 shows a graph of inhibition of histone acetyltransferase by various modified heparin oligosaccharides. Heparin with the sulfates removed from the 6 position of the glucoasamine (6-de), the 2 position of the uronic acid (2-de), or the N position of the glucosamine (N-de) were compared to each other, heparin, and chondroitin sulfate (Chondso4) in an In Vitro HAT activity assay. All samples were included in the assay at a concentration of 10 ug/ml.

FIG. 10 shows graph of inhibition of histone acetyltransferase by various sized oligosaccharides. Various sized oligosaccharides derived from heparin were tested at 10 ug/ml for HAT inhibitory activity in an In Vitro HAT activity assay: Tetra (4 sugars), Octa (8 sugars), Deca (10 sugars), Oligo II (12-14 sugars), Oligo I (14-18 sugars).

FIG. 11 shows a schematic of the procedure used to isolate and to prepare heparan sulfate proteoglycan ectodomains that lack chondroitin sulfate.

FIG. 12 shows In Vitro Inhibition of pCAF and p300 HAT activities by heparin. In the presence of 0.5 μCi [³H]acetyl Co A, 10 μg core histones was incubated with either 0.5 μg pCAF (filled circles, ) or 0.83 μg p300 HAT domain (open circles, ∘) and the indicated heparin concentrations for 30 minutes at 30° C. Formation of [³H]acetylated core histones was determined by vacuum filtration of the samples across a nitrocellulose membrane and quantitated by liquid scintillation counting. The data is expressed as the mean % Control ±SD. Background CPM in samples without added enzyme was 5781.25 while [³H]acetylated histone CPM in samples without heparin were 16484.5 for the pCAF containing samples and 9960.5 for the p300 HAT domain samples.

FIG. 13 shows inhibition of pCAF HAT Activity by other GAG classes. 10 μg core histones were incubated with 0.5 μg pCAF, 0.5 μCi [³H]acetyl Co A and the indicated concentrations of chondroitin sulfate (filled circles, ), dextran (open circles, ∘), D-glucosamine (filled squares, ▪), hyaluronic acid (open squares, □) or keratan sulfate (filled triangles, ▴) for 30 minutes at 30° C. 35 μl aliquots of the reaction mixtures were spotted onto nitrocellulose filter in a dot blot apparatus under vacuum to remove unincorporated [³H]acetyl Co A. The sample wells and filters were washed with 50 mM tris pH 7.6 and the samples were processed for liquid scintillation counting. The data is expressed as the mean % Control ±SD. 100% is equal to the activity in the absence of any additives.

FIG. 14 shows In Vitro inhibition of pCAF HAT activity by chemically modified heparin molecules. 10 μg core histones was incubated with 0.5 μg pCAF, 0.5 μCi [³H]acetyl Co A and the indicated concentrations of N-Desulfated Heparin (filled circles, ) or Fully O-Desulfated Heparin (open circles, ∘) for 30 minutes at 30° C. 35 μl aliquots of the reaction mixtures were filtered through a nitrocellulose filter in a Bio Dot Apparatus under vacuum. The wells and filter were washed with 50 mM tris pH 7.6 and the membrane was processed for liquid scintillation counting. The data is expressed as the mean % Control ±SD.

FIGS. 15A and 15B show inhibition of pCAF HAT Activity by elastase generated proteoglycans. FIG. 15A, Proteoglycans (PG) were purified from pulmonary fibroblast elastase supernatants using anion exchange chromatographic methods. Heparan sulfate proteoglycan fragments (HSPGf) were generated by treating the PG fraction with 10 mU/ml chondroitinase ABC and repurifying the fragments by anion exchange chromatography. Core histones (10 μg) were incubated with 0.5 μg pCAF, 0.5 μCi [³H]acetyl Co A and the indicated concentrations of PG (filled squares, ▪) or HSPGf (open squares, □) for 30 minutes at 30° C. 35 μl aliquots of the reaction mixtures were filtered through a nitrocellulose filter and the membrane was processed for liquid scintillation counting. The data is expressed as the mean % Control ±SD. FIG. 15B, Proteoglycans (PG) were purified from another preparation of pulmonary fibroblast elastase supernatants. Free GAG chains (B-PG) were generated by treating the PG fraction with alkaline borohydride and were recovered by anion exchange methods. Core histones (Core histones (10 μg) were incubated with 0.5 μg pCAF, 0.5 μCi [³H]acetyl Co A and the indicated concentrations of PG (filled squares, ▪) or B-PG (open squares, □) for 30 minutes at 30° C. 35 μl aliquots of the reaction mixtures were filtered through a nitrocellulose filter and the membrane was processed for liquid scintillation counting. The data is expressed as the mean % Control ±SD.

FIG. 16 shows that nuclear HSPG correlates with decreased cell growth rate. Pulmonary fibroblast were plated into 6-well plates and grown in media for the indicated number of days. Cells were labeled with ³⁵SO₄ (50 μCi/ml) starting on day 1 until time of extraction. At each time point, cell number was determined by measuring the level of acid phosphatase and relative growth rate (filled triangles, ▴) was calculated by determining the cell number difference between successive time points divided by the cell number at the preceding time point divided by the number of days. Nuclear HSPG levels (filled circles, ) were determined by zetaprobe analysis of nuclear extracts at each time point (see methods). All data represent the average ±SEM of six samples.

FIG. 17 shows heparin inhibits histone H3 acetylation in pulmonary fibroblasts and aortic smooth muscle cells. Primary neonatal rat fibroblasts and smooth muscle cells were established as described (Foster, J. A. et al., (1990)) and treated with heparin or N-desulfated heparin (10 μg/ml) for 24 h. Total cell extracts (100 μg protein for fibroblasts and 200 μg protein for smooth muscle cells) were generated and subjected to SDS-PAGE followed by electrotransfer to Immobilon membrane. Membranes were incubated with anti-acetylated histone H3 (Upstate) followed by enzyme linked secondary antibody. Bands were visualized with ECL.

FIG. 18 shows Iii Vitro inhibition of pCAF HAT activity by chemically modified heparin molecules. 10 μg core histones was incubated with 0.5 μg pCAF, 0.5 μCi [³H]acetyl Co A and the indicated concentrations of 2-O-Desulfated Heparin () or 6-O-Desulfated Heparin (∘) for 30 minutes at 30° C. 35 μl aliquots of the reaction mixtures were filtered through a nitrocellulose filter in a Bio Dot Apparatus under vacuum. The wells and filter were washed with 50 mM tris pH 7.6 and the membrane was processed for liquid scintillation counting. The data is expressed as the mean % Control ±SD.

FIG. 19 shows the effect of Glucuronic acid and N-Acetyl-D-glucosamine in a pCAF histone acetylation assay. 10 μg core histones was incubated with 0.5 μg pCAF, 0.5 μCi [³H]acetyl Co A and the indicated concentrations of Glucuronic Acid (⋄) and N-Acetyl-D-Glucosamine (♦ for 30 minutes at 30° C. 35 μl aliquots of the reaction mixtures were spotted onto nitrocellulose filter in a dot blot apparatus under vacuum to remove unincorporated [³H]acetyl Co A. The sample wells and filters were washed with 50 mM tris pH 7.6 and the samples were processed for liquid scintillation counting. The data is expressed as the mean % Control ±SD.

### DETAILED DESCRIPTION OF THE INVENTION

We have discovered that glycosaminoglycans are potent inhibitors of histone acetyltransferases (HAT'S) that can be used in methods for inhibition of histone acetyltransferases. Preferably one uses heparin and heparan sulfate oligosaccharides. Most preferably one uses modified heparin, heparan sulfate (HS), or heparan sulfate proteoglycan (HSPG) that contains sulfation at the N-position of glucosamine residues and lacks O-sulfation on the uronic acid and/or glucosamine residues. In one embodiment, the invention is further directed to methods for treating disorders associated with hyperacetylation by administration of a compound containing glycosaminoglycans (e.g. heparin or heparan sulfate oligosaccharide) as an active ingredient to a patient in need thereof.

### Glycosaminoglycans

Glycosaminoglycans belong to a highly heterogeneous class of macromolecules and are long molecules containing repeating disaccharide units forming linear macromolecules. In general each of the repeating units comprises a residue consisting of an aminosugar, that is glucosamine or galactosamine, and a uronic acid residue consisting of glucuronic acid or iduronic acid. The hydroxyl group at C (2), C (3), C (4) and C (6) and the amino group on C(2) may be substituted by sulfate groups. GAGs include the following compounds: heparin, heparan sulfate (HS), dermatan sulfate (DS), hyaluronic acid (HA), chondroitin sulfate (CS), and keratan sulfate.

Generally, in nature, a glycosaminoglycan (GAG), is covalently attached to a protein core which often contains other glycosaminoglycans, e.g. a protein core may contain both heparan sulfate and chondroitin sulfate (Williams and Fuki 1997). Hyaluronic acid is not attached to a protein core.

Heparin and Heparan Sulfate Oligosaccharides

Heparan sulfate (HS) is a linear oligosaccharide that, in nature, is covalently attached to a protein core which often contains other glycosaminoglycans. When the protein core contains heparan sulfate, the entire molecule is referred to as a heparan sulfate proteoglycan (HSPG). Core proteins vary in size from 32 to 500 kDa.

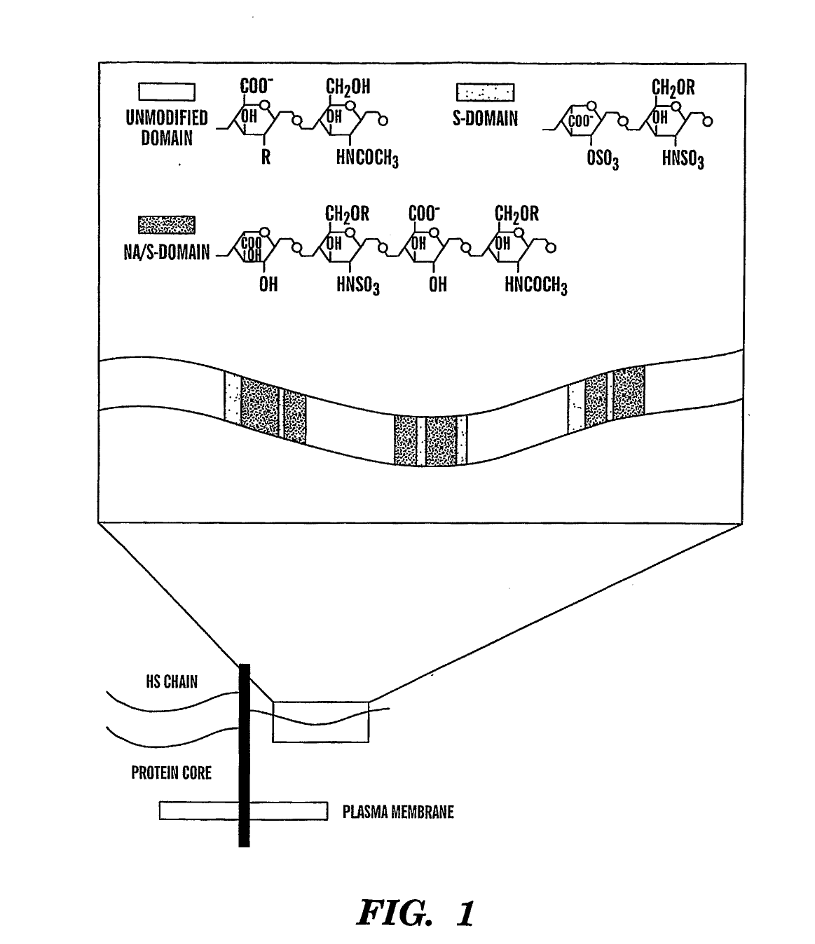

Heparan sulfate macromolecules consist of 50-200 repeating disaccharide units (25-100 kDa). These disaccharide units consist of glucuronic acid (GlcA) or iduronic acid (IdoA) α-linked to N-acetylglucosamine (GlcNAc). Biosynthesis of HS occurs in the Golgi apparatus and is a complex process that begins with the stepwise addition of a xylose, two galactose, and a GlcA to a serine residue on the core protein. Subsequently, GlcNAc is added committing the chain to HS synthesis. Following polymerization, a series of enzyme reactions results in regions of variable sulfation and acetylation (FIG. 1). The exact pattern of these modifications can vary greatly between HS chains, and it is this variation that allows the many binding and regulatory properties of HS towards proteins (Turnbull et al. 2001).

Heparin is a molecule closely related to heparan sulfate as heparin also comprises polymers of repeating disaccharide units; D-glucosamine-L-iduronic acid and D-glucosamine-D-glucuronic acid. However, heparin contains relatively more iduronic acid than heparan sulfate and has a higher degree of sulfation.

Low molecular weight heparins have a Mr of between 2 and 10 kDa. They can be prepared from heparins by specific chemical cleavage and typically contain the anticoagulant pentasaccharide. Their main clinical function is to inhibit factor Xa, resulting in an antithrombotic effect. LMW heparins are also proposed to have antimetastatic properties. Heparin fragments having selective anticoagulant activity, as well as methods for the preparation thereof, are described in U.S. Pat. No. 4,303,651. However, having the anticoagulant effect is generally not desirable for the methods described herein.

Ultra-low molecular weight heparins have a molecular weight less than 3,000 daltons. In one embodiment, the methods of the invention do not include the use of ultra-low molecular weight heparins having an average molecular weight of less than 3 kDa.

In one preferred embodiment, one uses oligosaccharides (e.g. heparin, heparan sulfate, or HSPG) that have been chemically or enzymatically modified so that the specific sulfation pattern has been altered (i.e. oligosaccharides where sulfate is chemically removed from the N-position of the glucosamine residues, or from the 2-0 position of the iduronic/glucoronic acid residue, or from the 6-0 position of the glucosamine, or from the 3-0 position of the glucosamine). Preferably, the oligosaccharide contains sulfation at the N-position of the glucosamine residues and sulfation is removed from either the 2-0 position of the iduronic/glucoronic acid residue, or from the 6-0 position of the glucosamine.

It is preferred that the GAGs including heparin do not have anticoagulant activity. This can be accomplished by known means such as deleting the domain responsible for anticoagulant activity or disrupting that domain so that the molecule does not display anticoagulant activity. This anticoagulant activity can be defined by the absence of antithrombin III binding activity.

Compounds with the desired properties can be obtained from heparin and heparan sulfate fractions using specific periodate oxidation to eradicate the antithrombin III binding properties. Selective N-desulfation followed by re-N-acetylation, or selective O— desulfation also yields compounds with low anticoagulant activity. In addition, selective N-deacetylation followed by specific N- and/or O-sulfation yields compounds of desired activity.

In another preferred embodiment, one can use a portion based upon cell surface HSPG ecto-domain fragments (HSPG_{f}).

### Chrondroitin Sulfate, Dermatan Sulfate and Keratan Sulfate Oligosaccharides

Chondroitin sulfate (CS) is a sulfated linear polysaccharide consisting of alternating glucuronic acid and N-acetyl-galactosamine residues, the latter being sulfated in either 4 or 6 position. They can be prepared from bovine tracheal or nasal cartilage. CS is of importance for the organization of extracellular matrix, generating a interstitial swelling pressure and participating in recruitment of neutrophils.

In one embodiment, chondroitin sulfates and derivatives are used in methods of the invention.

Dermatan sulfate (DS) is a sulfated linear polysaccharide consisting of alternating uronic acid and N-acetylated galactosamine residues. The uronic acids are either D-GlcA or L-IdoA and the disaccharide can be sulfated in 4 and 6 and 2 on galactosamine and IdoA, respectively. DS can be prepared from porcine skin and intestinal mucosa. Dermatan sulfate possesses biological activities such as organization of extracellular matrix, interactions with cytokines, anti-coagulant activities and recruitment of neutrophils. Again, it is preferred that the protein is modified to remove anticoagulant activity.

In one embodiment, dermatan sulfates and derivatives are used in methods of the invention.

Keratan sulfate is a glycosaminoglycan having N-acetyllactosamine as the basic structure which has O-sulfated hydroxyl group at C-6 position of the N-acetylglucosamine residue. Especially, high-sulfated keratan sulfate which further has a sulfated hydroxyl group beside that at C-6 position of N-acetylglucosamine residue in the constitutional disaccharide unit is known to be contained in cartilaginous fishes such as sharks, and cartilage, bone and cornea of mammals such as whale and bovines.

In one embodiment, keratan sulfates and derivatives are used in methods of the invention.

### Hyaluronic Acid

Hyaluronan (also known as hyaluronic acid or hyaluronate) (HA), is a glycosaminoglycan lacking a protein core, and is one of the major non-structural elements of the extracellular matrix. HA also is expressed on cell surfaces and has been shown to bind several different molecules, including CD44.

HA is a repeating disaccharide of alternately linked residues of glucuronic acid (GlcA) and N-acetylglucosamine (GlcNAc). HA that exists in vivo as a high molecular weight linear polysaccharide and is found in mammals predominantly in connective tissues, skin, cartilage, and in synovial fluid, and is also the main constituent of the vitreous of the eye. In connective tissue, the water of hydration associated with HA creates spaces between tissues, thus creating an environment conducive to cell movement and proliferation. HA plays a key role in biological phenomena associated with cell motility including rapid development, regeneration, repair, embryogenesis, embryological development, wound healing, angiogenesis, and tumorigenesis (Toole et al. Plenum Press, New York, 1384-1386, 1991; Bertrand et al. Int. J. Cancer. 52:1-6, 1992; Knudson et al. F.A.S.E.B. J. 7:1233-1241, 1993). HA levels have been shown to correlate with tumor aggressiveness (Ozello et al. Cancer. Res. 20:600-604, 1960; Takeuchi et al. Cancer. Res. 36:2133-2139, 1976; Kimata et al. Cancer. Res. 43:1347-1354, 1983), and can be indicative of the invasive properties of tumor cells (Knupfer et al. Anticancer. Res. 18:353-6, 1998).

HA also is involved in immune responses, for example, increased binding of HA to one of its receptors, CD44, has been shown to mediate the primary adhesion (“rolling”) of lymphocytes to vascular endothelial cells under conditions of physiologic shear stress, and this interaction mediates activated T cell extravasation into an inflamed site in vivo in mice (DeGrendele et al. J. Exp. Med. 183:1119-1130, 1996; DeGrendele et al., J. Immunol. 159:2549-2553, 1997; DeGrendele, et al., Science. 278:672-675, 1997b).

In one embodiment, hyaluronates and derivatives are used in methods of the invention.

Also contemplated are the use of derivatives of the above identified glycosaminoglycans. Derivatives include glycosaminoglycans that have been subjected to chemical and enzymatic modification, for example to remove or add sulfation and anticoagulant activity or to generate oligosaccharides of specified length.

We have determined that heparin/heparan sulfate and chondroitin sulfate are more potent inhibitors of histone acetyltransferase than hyaluronic acid. Accordingly, a preferred embodiment of the invention comprises the use of heparin, heparan sulfate, or chondroitin sulfate and derivatives thereof. More preferably, the methods of the invention comprise the use of heparin and heparan sulfate, as heparin and heparan sulfate are more potent inhibitors of histone acetyltransferase than chondroitin sulfate.

In one preferred embodiment, a heparan sulfate proteoglycan ectodomain is used as an inhibitor of histone acetyltransferase. Preferably, the heparan sulfate proteoglycan ectodomain is derived from or equivalent to that derived form corneal stromal fibroblasts, or from pulmonary fibroblasts.

There are numerous reports describing the nuclear localization of GAGs such as HS and HSPG, which collectively suggest specific roles for these molecules in transcriptional regulation. For example, the nuclear localization pattern of glypican in neurons and glioma cells has been shown to change with different phases of the cell cycle (Liang et al. 1997). In addition, specific HS structure may be important in the regulation of cell cycle progression by nuclear HSPG. Fedarko, Conrad, and Ishihara have shown that HS enriched in sulfated glucuronic acid (GlcA) residues accumulate in the nucleus of a rat hepatocyte cell line (Fedarko and Conrad 1986; Ishihara et al. 1986). Furthermore, Fedarko et al. showed that the nuclear localization of HSPG isolated from log phase vs. confluent hepatoma cell cultures had different effects on cell cycle progression, further suggesting that specific HS moieties are important in regulating cell growth (Fedarko et al. 1989). This regulation may involve the ability of HS to inhibit specific transcription factors from interacting with their consensus oligonucleotide elements (Dudas et al. 2000). Kovalszky reports that heparin and HS from normal liver, but not from its malignant counterpart, inhibit DNA topoisomerase I activity in nuclear extracts of malignant cell lines (Kovalszky et al. 1998).

Nuclear localization of GAGs, such as HS or HSPG, have been described in other systems as well. In human lung fibroblasts, an L-iduronate rich species of HS is internalized and its anti-proliferative effects correlate with its appearance in the nucleus (Arroyo-Yanguas et al. 1997; Cheng et al. 2001). Another body of evidence that suggests HSPG may have specific functions in the nucleus stems from the investigation of autoimmune diseases such as systemic lupus erythematosus (SLE), where antibodies against nuclear material are found. In these studies, HSPG was stated to bind nucleosomes, perhaps through an ionic interaction (Watson et al. 1999), and this mechanism might be important for chromatin clearance (Du Clos et al. 1999). In addition, cell surface HSPG have been shown to mediate the infection of a number of viruses that include human immunodeficiency virus (HIV), herpes simplex virus type I (HSV-1), and human cytomegalovirus (HCMV) (Patel et al. 1993; Immergluck et al. 1998; Song et al. 2001).

Recently, HSPG has been shown to localize to the nucleus in corneal stromal fibroblasts adherent to FN but not CO (Richardson et al. 2000). The significance of its translocation to the nucleus is not understood but certain possibilities exist. For example, HSPG may function to transport heparin-binding proteins, such as certain growth factors, to the nucleus where these proteins can subsequently directly influence transcriptional events. Secondly, HSPG itself may regulate nuclear activities related to the wound healing process.

Several reports suggest that HSPGs localize to the nucleus and may directly modulate gene expression by interacting with nuclear machinery through their HS chains, but specific mechanisms have not been elucidated (Fedarko and Conrad 1986; Ishihara et al. 1986; Fedarko et al. 1989; Liang et al. 1997; Rykova and Grigorieva 1998; Cheng et al. 2001). We have found a new role for GAGs, such as heparin sulfate and HSPG, particularly in the nucleus; inhibition of histone acetyltransferase activity.

### Sequence Specificity of Heparan Sulfate

Heparin and heparan sulfate are highly heterogeneous molecules. The repeating disaccharide unit of heparin and heparan sulfate is comprised of alternating glucosamine and hexuronic acid monosaccharides. The hexuronic acid of heparin or heparan sulfate can be either glucuronic acid or iduronic acid (glucuronic acid that has undergone C5 epimerization of the carboxyl group). Heparin only differs from heparan sulfate in that it contains relatively more iduronic acid, N—, and O-sulfation (for a review, see generally, R. L. Jackson et al., (1991) Physiological Reviews 71:481).

Heterogeneity in GAGs results from variations in chain length, different carbohydrate backbone sequences, and the pattern and degree of sulfation. Recent studies have indicated that specific regions or “sequences” along heparan sulfate chains allow for high affinity binding and modulation of a wide range of enzymes, hormones, and growth factors (Nugent, PNAS 97(19):10301-10303 (2000)).

The GAGs such as heparin and heparan sulfate oligosaccharides of the invention can be obtained from natural sources. Alternatively, synthetic oligosaccharides or biomimetic chemicals can be used in place of naturally derived GAGs, e.g. heparan sulfates. Means for isolation, identification, and quantitation of specific GAGs are well known to those skilled in the art.

Preferably, GAGs such as heparin and heparin sulfate oligosaccharides of a specific sequence are used to inhibit histone acetyltransferase. Isolated or synthetic oligosaccharides can be modified chemically or enzymatically by means known in the art, for example to remove sulfation or acetylation on specific residues.

In one embodiment, the oligosaccharides are of at least 5, 6, or 7 sugars in length.

In one embodiment, the oligosaccharides are of at least 8-12 sugars in length and contain N-sulfated glucosamine residues. More preferably the oligosaccharide contains O-sulfation at either the 6-O or 2-O position, but not at both positions.

Specific activity of the oligosaccharide chains as inhibitors of histone acetyltransferase activity can be assayed as described in the examples herein or by methods as described in U.S. patent application 200100910967, which is herein incorporated by reference in its entirety.

### Histone Acetyltransferases

Acetylation involves the reversible modification of lysine residues. Many interactions between proteins and HS involve the coordination of positively-charged lysine residues with negatively-charged sulfate groups (Gregory et al. 2001). Chromatin remodeling by acetylation is an important component of gene expression, and the identification of histone acetyltransferases (HAT) has led to further insight into how these enzymes effect transcription (Brown et al. 2000; Sterner and Berger 2000; Gregory et al. 2001; Marmorstein and Roth 2001). Although histone acetylation has been correlated with transcriptional activation for over 30 years, the first transcription-related HAT was discovered in 1996 (Brownell et al. 1996).

There are now five reported families of acetyltransferases, comprising over twenty enzymes, which generate specific patterns of free and/or nucleosome-associated histone acetylation. These include the 1) GNAT superfamily (Gcn5-related N-acetylransferases), which includes proteins involved with, or linked to, transcriptional initiation (Gcn5 and PCAF), elongation (Elp3), histone deposition and telomeric silencing (Hat1); 2) the MYST family named after the founding members MOZ, Ybf2/Sas3, Sas2 and Tip60; 3) the p300/CBP HAT family, comprised of the highly related p300 and CBP proteins, which share sequence homology with GNATs; 4) the p300/CBP family, which have been extensively described as coactivators for multiple transcription factors and includes the TFIID subunit TAF250; and 5) the nuclear hormone-related HATs SRC1 and ACTR (SRC3). See, Timmermann et al., cellular and Molecular Life Sciences 58: 728-276 (2001); Kawahara et al., Ageing Research Reviews, 2: 287-297 (2203): and Carrozza et al. Trends in Genetics, 19 (6): 321-329 (2003), which are herein incorporated by reference.

Any HAT can be inhibited by methods of the invention. Specific examples of HATS that can be inhibited by methods of the invention include, but are not limited to, hTAFII250, TFIIIC220, TFIIIC10, TFIIIC90, hHat1, hGcn5-L, pCAF, CBP/p300, SRC-1, ACTR (RAC3, TRAM1, AIB1, p/CIP), HBO1, MORF (NOZ), and Tip60.

In one embodiment, the HAT is p300.

In one embodiment the HAT is pCAF.

The HAT activity of p300 and CBP is required for their role in transactivation, and these enzymes have been found to associate with other acetyltransferases, indicating that multiple HAT enzymes may be recruited to act cooperatively during gene activation.

Hyperacetylation of histones and other proteins modified by HATs affects cellular proliferation, differentiation and apoptosis which can lead to a variety of disorders. Disorders associated with hyperacetylation include, but are not limited to, cancers, cardiovascular disease, proliferative eye disease (diabetic retinopathy), psoriasis, arthritis and chronic obstructive pulmonary disease.

The invention provides methods for treatment of disorders associated with hyperacetylation by administering a composition containing a glycosaminoglycan (e.g. heparin or heparan sulfate oligosaccharides) as the active agent to a patient in need thereof. Any disorder that has as a characteristic hyperacetylation can be treated by methods of the invention.

Histone acetylation and deacetylation are important factors in inflammatory lung diseases such as cystic fibrosis, chronic obstructive pulmonary disorder (COPD), interstitial lung disease and acute respiratory distress syndrome (Barnes et al. Eur Respir J. 25(3):552-63 (2005)). Further, it has recently been shown the increased inflammatory response seen in asthma corresponds to a reduction in HDAC activity and increase in HAT activity (Cosio et al., Am. J. Respir. Crit. Care Med. 170: pp 141-147 (2004)).

In one embodiment, chronic obstructive pulmonary disorder (COPD) is treated by methods of the invention. Chronic obstructive pulmonary disorder (COPD) also referred to as chronic obstructive pulmonary disease refers to a group of disorders that damage the lungs and make breathing increasingly more difficult over time. Common COPD's include chronic bronchitis, emphysema and asthma.

In one embodiment, asthma is treated by methods of the invention.

In one embodiment, the late stages of asthma (after antigen challenge) are treated using the methods of the invention. Preferably, heparan sulfate glycosaminoglycans or heparin are used for treating late stages of asthma. In one embodiment, the heparin sulfate glycosaminoglycan or heparin is not less than 3,000 daltons.

In one embodiment, chronic bronchitis is treated by methods of the invention.

In one embodiment, emphysema is treated by methods of the invention.

Cancers that can be treated by methods of the invention include, but are not limited to, breast cancer, basal cell carcinoma, gastrointestinal cancer, lip cancer, mouth cancer, esophageal cancer, small bowel cancer and stomach cancer, colon cancer, liver cancer, bladder cancer, pancreas cancer, ovary cancer, cervical cancer, lung cancer, breast cancer and skin cancer, such as squamous cell and basal cell cancers, prostate cancer, renal cell carcinoma, as well as other known cancers that effect epithelial cells throughout the body, and cancers of hematopoietic origin such as leukemia.

Hyper-nuclear-acetylation has also been linked with a variety of cardiovascular disorders and rheumatoid arthritis. For example, CBP histone acetylase is responsible for hyperacetylation in atherosclerotic lesions and is associated with hyperacetylation in rheumatoid arthritis synovium and cultured synoviocytes (Kawahara et al., Ageing Research Reviews 2: 287-297 (2003)).

In one embodiment, cardiac disorders are treated by methods of the invention. A preferred cardiac disorder to be treated is atherosclerosis.

In one embodiment, the cardiac disorder to be treated is not restenosis.

HAT activity has further been linked to cell differentiation. Thus, heparan sulfate/heparin oligosaccharide inhibitors described herein can also be used to induce differentiation of stem cells to a desired fate.

### Administration

The invention encompasses the preparation and use of pharmaceutical compositions comprising the glycosaminoglycan (e.g. heparin/heparan sulfate, hyaluronate, and chondroitin sulfate oligosaccharides) of the invention as an active ingredient. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. Administration of one of these pharmaceutical compositions to a subject is useful for treating a variety of diseases or disorders as described elsewhere herein. The active ingredient may be present in the pharmaceutical composition in the form of a physiologically acceptable ester or salt, such as in combination with a physiologically acceptable cation or anion, as is well known in the art.

As used herein, the term “pharmaceutically acceptable carrier” means a chemical composition with which the active ingredient may be combined and which, following the combination, can be used to administer the active ingredient to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs, birds including commercially relevant birds such as chickens, ducks, geese, and turkeys.

Pharmaceutical compositions that are useful in the methods of the invention may be prepared, packaged, or sold in formulations suitable for oral, rectal, vaginal, parenteral, topical, pulmonary, intranasal, buccal, ophthalmic, or another route of administration, for example continuous infusion via pumps or by implantable controlled release systems that can deliver the pharmaceutical composition locally. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. For example, oligosaccharides can be mixed to target multiple proteins. Controlled- or sustained-release formulations of a pharmaceutical composition of the invention may be made using conventional technology.

A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

As used herein, an “oily” liquid is one which comprises a carbon-containing liquid molecule and which exhibits a less polar character than water.

A tablet-comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically-controlled release tablets. Tablets may further comprise a sweetening agent, a flavoring agent, a coloring agent, a preservative, or some combination of these in order to provide pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

A pharmaceutical composition of the invention may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e. about 20° C.) and which is liquid at the rectal temperature of the subject (i.e. about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants and preservatives.

Retention enema preparations or solutions for rectal or colonic irrigation may be made by combining the active ingredient with a pharmaceutically acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants and preservatives.

A pharmaceutical composition of the invention may be prepared, packaged, or sold in a formulation suitable for vaginal administration. Such a composition may be in the form of, for example, a suppository, an impregnated or coated vaginally-insertable material such as a tampon, a douche preparation, or a solution for vaginal irrigation.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e. such as with a physiologically degradable material), and methods of absorbing an aqueous or oily solution or suspension into an absorbent material, with or without subsequent drying.

Douche preparations or solutions for vaginal irrigation may be made by combining the active ingredient with a pharmaceutically acceptable liquid carrier. As is well known in the art, douche preparations may be administered using, and may be packaged within, a delivery device adapted to the vaginal anatomy of the subject. Douche preparations may further comprise various additional ingredients including, but not limited to, antioxidants, antibiotics, antifungal agents, and preservatives.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, continuous infusion of the composition, by application of the composition through a surgical incision, by application of the composition through a tissue-penetrating non-surgical wound, and the like. Compositions may be delivered by controlled release systems, such as patches or polymer-based systems. In particular, parenteral administration is contemplated to include, but is not limited to, subcutaneous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition. These formulations may be sold as kits. For example, in dry form for reconstitution with instructions for use.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi-liquid preparations such as liniments, lotions, oil-in-water or water-in-oil emulsions such as creams, ointments or pastes, and solutions or suspensions. Topically-administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

A pharmaceutical composition of the invention may be prepared, packaged, or sold in a kit formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65 degrees F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold in kits as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to 500 micrometers. Such a formulation is administered in the manner in which snuff is taken i.e. by rapid inhalation through the nasal passage from a container of the powder held close to the nares.

Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

A pharmaceutical composition of the invention may be prepared, packaged, or sold in a formulation suitable for ophthalmic administration. Such formulations may, for example, be in the form of eye drops including, for example, a 0.1-1.0% (w/w) solution or suspension of the active ingredient in an aqueous or oily liquid carrier. Such drops may further comprise buffering agents, salts, or one or more other of the additional ingredients described herein. Other opthalmalogically-administrable formulations which are useful include those which comprise the active ingredient in microcrystalline form or in a liposomal preparation.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, Pa., which is incorporated herein by reference.

The oligosaccharides of the invention can also be linked to peptides or other agents to increase the targeting to desired cells and tissues or to enhance targeting to the nucleus within the cell. For example, the bioavailability can be enhanced through the use of cationic and peptoid based excipients (Malkove et al., Pharm, Res, 19: 1180-1184 (2002)).

In one preferred embodiment, the oligosaccharide is complexed with a polyaminoester, for example poly(β-amino ester) (Linhardt, Chemistry and Biology 11: 420-422 (2004); Lynn & Langer, J. Am. Chem. Soc. 122, 10761-10768 (2000); Berry et al. Chemistry and Biology 11: 487-498 (2004)).

The determination of a therapeutically effective dose is well within the capability of those skilled in the art. A therapeutically effective dose refers to that amount of active ingredient which decreases histone acetyltransferase activity relative to the histone acetyltransferase activity which occurs in the absence of the therapeutically effective dose.

For any oligosaccharide, the therapeutically effective dose can be estimated initially either in cell culture assays or in animal models, usually mice, rabbits, dogs, or pigs. The animal model also can be used to determine the appropriate concentration range and route of administration. Such information can then be used to determine useful doses and routes for administration in humans.

Therapeutic efficacy and toxicity, e.g., ED₅₀ (the dose therapeutically effective in 50% of the population) and LD₅₀ (the dose lethal to 50% of the population), can be determined by standard pharmaceutical procedures in cell cultures or experimental animals. The dose ratio of toxic to therapeutic effects is the therapeutic index, and it can be expressed as the ratio, LD₅₀/ED₅₀.

Pharmaceutical compositions that exhibit large therapeutic indices are preferred. The data obtained from cell culture assays and animal studies is used in formulating a range of dosage for human use. The dosage contained in such compositions is preferably within a range of circulating concentrations that include the ED₅₀ with little or no toxicity. The dosage varies within this range depending upon the dosage form employed, sensitivity of the patient, and the route of administration.

The exact dosage will be determined by the practitioner, in light of factors related to the subject that requires treatment. Dosage and administration are adjusted to provide sufficient levels of the active ingredient or to maintain the desired effect. Factors that can be taken into account include the severity of the disease state, general health of the subject, age, weight, and gender of the subject diet, time and frequency of administration, drug combination(s), reaction sensitivities, and tolerance/response to therapy.

Dosage amounts can range from 0.1 to 100,000 micrograms, up to a total dose of about 10 g, depending upon the route of administration. While the precise dosage administered will vary depending upon any number of factors, including but not limited to, the type of animal and type of disease state being treated, the age of the animal and the route of administration. Preferably, the dosage of the compound will vary from about 1 mg to about 10 g per kilogram of body weight of the animal. More preferably, the dosage will vary from about 10 mg to about 1 g per kilogram of body weight of the animal.

The compound may be administered to an animal as frequently as several times daily, or it may be administered less frequently, such as once a day, once a week, once every two weeks, once a month, or even less frequently. Alternatively, the composition can be delivered continuously. The frequency of the dose will be readily apparent to the skilled artisan and will depend upon any number of factors, such as, but not limited to, the type and severity of the disease being treated, the type and age of the animal, etc.

The invention is now described with reference to the following experimental details. The experimental details are provided for the purpose of illustration only and the invention should in no way be construed as being limited to the embodiments described herein, but rather should be construed to encompass any and all variations which become evident as a result of the teaching provided herein.

### EXAMPLES

### Example 1

### HSPG Inhibits Histone Acetyltransferase Activity In Vitro

Materials and methods

### In Vitro HAT Activity Assay

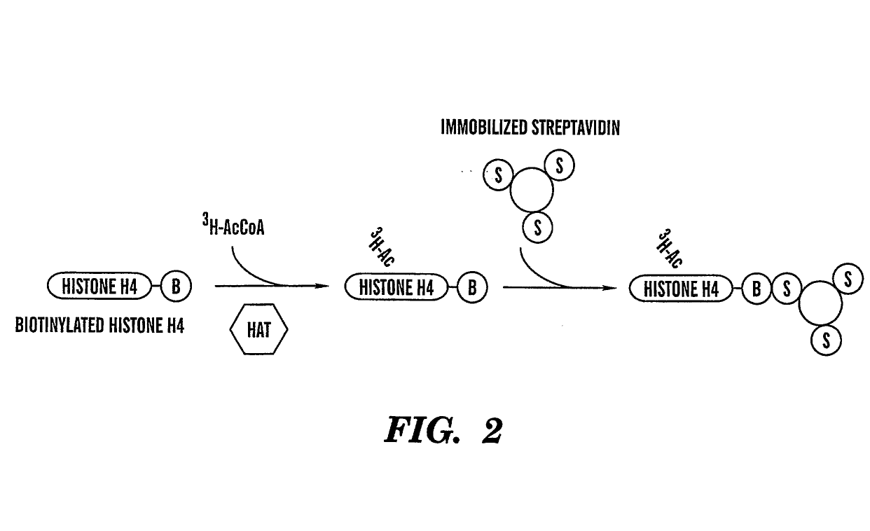

HAT activity was assessed in vitro as outlined in FIG. 2. In a 1.7 mL microcentrifuge tube, 100 μL of substrate (biotinylated histone H4 peptide in 50 mM Tris, pH 7.4, 1 mM EDTA; from Pierce), 100 μL of a 5 mg/mL BSA solution (in H2O), 10.5 μL of 10×HAT buffer (500 mM Tris, pH 7.4, 10 mM EDTA), 20 U/mL of HAT enzyme (p300 HAT domain; from Upstate), and 1 μCi/mL of [³H] Acetyl-CoA (Amersham) were mixed together and the reaction was allowed to proceed at 30° C. for 1 hour. Following this incubation, 100 μL of a 1:1 slurry of immobilized streptavidin (Pierce) pre-equilibrated in 1×HAT buffer (50 mM Tris, pH 7.4, 1 mM EDTA) was added and the sample was incubated for 30 min at RT on a rotating platform. After centrifugation at 10,000 g for 4 min, the supernatant containing the excess reactants was removed and the pellet containing the acetylated substrate bound to the immobilized streptavidin was washed 3 times with 500 μL RIPA buffer (50 mM Tris, pH 7.4, 150 mM NaCl, 1 mM EDTA, 1% NP-40, 0.5% SDC, 0.1% SDS). After the final wash, the pellet was resuspended in 500 μL 1×HAT buffer, diluted in EcoLite, and counted in a scintillation counter. Because of sample volume restrictions, reaction volumes were scaled down in some experiments.

### Extraction of Cell Surface HSPG Ectodomain by Mild Trypsin Digestion

Trypsin releases a heparan sulfate-rich ectodomain from cell surface proteoglycans (Rapraeger and Bernfield 1985). Confluent monolayers were rinsed twice with DPBS (Gibco/Invitrogen) and scraped into 1 mL extraction buffer (DPBS w/o CaCl2, MgCl2, 0.5 mM EDTA) containing 0.5 mM PMSF, 50 μg/mL soybean trypsin inhibitor (Sigma), 5 mM N-ethylmaleimide (Sigma), 1 μM Pepstatin A. The cells were washed four times with extraction buffer by centrifugation (200×g; 2 min) and resuspended in 1 mL cold extraction buffer. Washed cell suspensions were incubated in a final concentration of 20 μg/mL bovine pancreatic trypsin (Sigma) for 5 min on ice. To stop the reaction, soybean trypsin inhibitor was added to a final concentration of 200 μg/mL, followed by centrifugation (200×g; 2 min). The supernatant, containing the trypsin-released HSPG ectodomain, was collected and stored at −20° C. until purification by Q-sepharose chromatography could be performed.

Purification of proteoglycan Fractions Using Q-Sepharose Chromatography

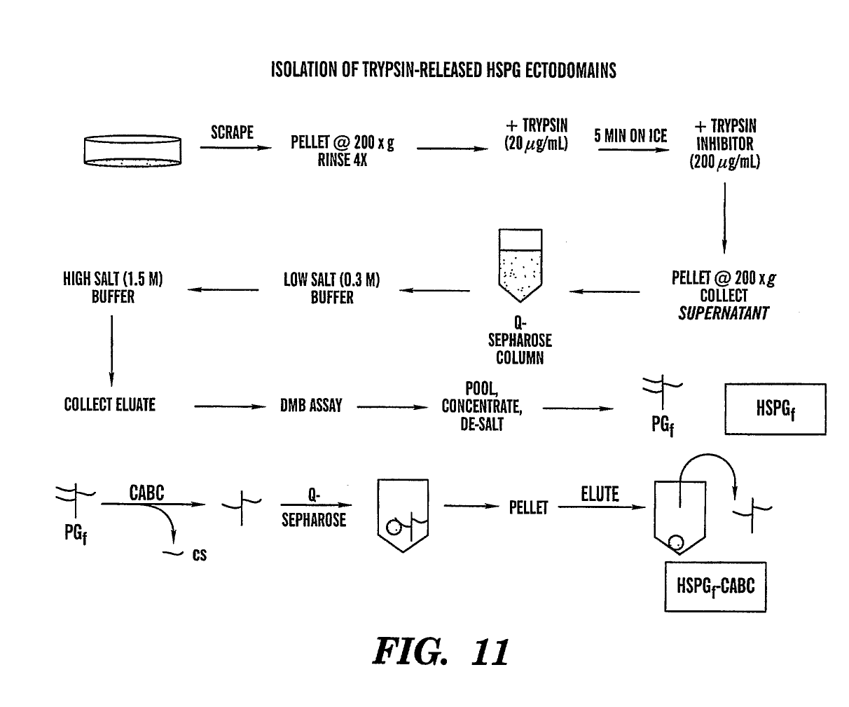

The purification of trypsin-released HSPG ectodomains was adapted from a previously described method (Brown et al. 2002), see FIG. 11. Extracts were diluted 1:1 in freshly prepared 2× Q1 buffer (100 mM sodium acetate, pH 6.0, 600 mM NaCl, 20 mM EDTA, 40% propylene glycol) and filtered through a 0.22 μm polyethylenesulfone (PES) filter (Corning). Diluted extracts were loaded onto a 15 mL Q-Sepharose column equilibrated with Q1 buffer (50 mM sodium acetate, pH 6.0, 300 mM NaCl, 10 mM EDTA, 20% propylene glycol) and the column was washed with Q1 buffer until the UV absorbance at 280 nm (A280) decreased to baseline. The conductivity and A280 were monitored during the entire process. The column was washed with five column volumes of Low Salt Buffer (50 mM sodium acetate, pH 6.0, 300 mM NaCl). Proteoglycans were eluted with High Salt Buffer (50 mM sodium acetate, pH 6.0, 1.5 M NaCl) and fractions were collected. Fractions were analyzed using the DMB assay. GAG containing fractions were pooled, de-salted into PBS, and concentrated in a Centricon YM-10 centrifugal filter device (Millipore). Concentration of sulfated GAG was determined by DMB assay.

To determine the composition and role of specific GAG, purified trypsin-released HSPG fragments (HSPGf) were further digested with CABC (5 mU/mL), heparinase 1 (2 μg/mL), or heparinase III (0.1 U/mL) in Q1 buffer for 1 hr at 37° C. Upon confirmation of a successful digestion by DMB assay, digested HSPGf was incubated with a small amount of Q-Sepharose resin at 4° C. overnight, and pelleted at 1000 g for 10 min. The pellet was washed once with Q1 buffer and once with Low Salt buffer. Lyase-digested HSPGf were eluted with three volumes of High Salt buffer (50 mM sodium acetate, pH 6.0, 3 M NaCl). High Salt buffer washes were collected and pooled. Pooled washes were de-salted into PBS and concentrated in a Centricon YM-10 centrifugal filter device. Final GAG concentration was determined by DMB assay, see FIG. 11.

### Heparin Inhibits HAT Activity In Vitro

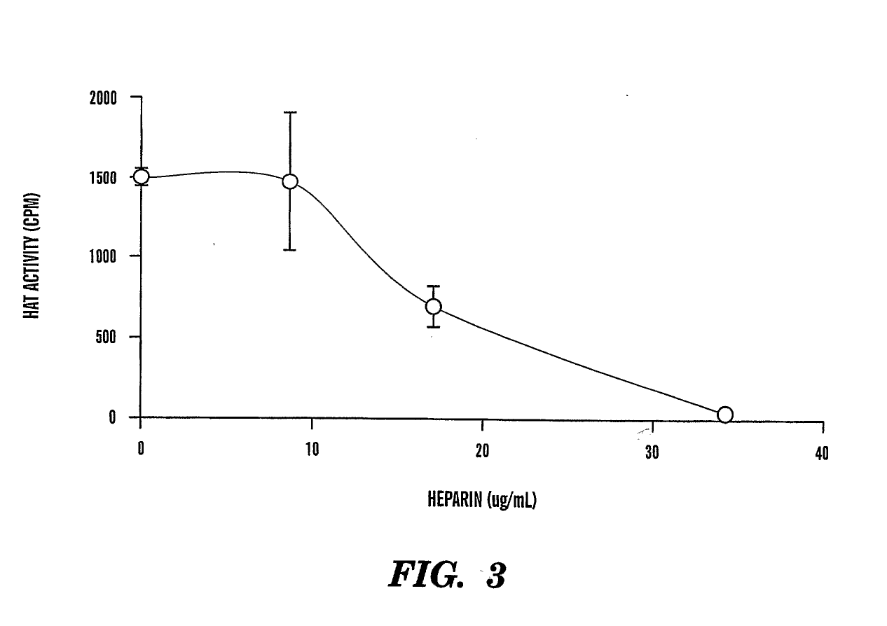

Heparin is a unique subclass of HS synthesized in mast cells and some other mammalian cells that is more extensively modified than HS. While heparin is confined to mast cells, where it is stored in cytoplasmic granules, HS is ubiquitously distributed on cell surfaces and in extracellular matrices. Heparin is generally more sulfated (>80% of glucosamine residues are N-sulfated and the concentration of O-sulfate groups exceeds that of N-sulfate groups), whereas HS contains regions of desulfation interspersed between highly sulfated (heparin-like) regions (Roden et al. 1992; Salmivirta et al. 1996; Sugahara and Kitagawa 2002). To study the effects of heparin on HAT activity, we employed an in vitro assay. Biotinylated histone H4 peptide (substrate) was incubated with recombinant p300 HAT enzyme and [³H]-acetyl CoA in the presence of increasing concentrations of heparin. Immobilized streptavidin was used to capture the modified substrate and was counted in a scintillation counter (FIG. 3). HAT activity, as measured by levels of [³H]-acetylated substrate, decreased as the concentration of heparin increased, suggesting that heparin inhibited this reaction. A 50% reduction was seen with 17 μg/mL heparin, while 34 μg/mL resulted in almost complete inhibition. Thus, the inhibition of HAT by heparin suggests that similar molecules, such as HS, may also be capable of inhibiting HAT.

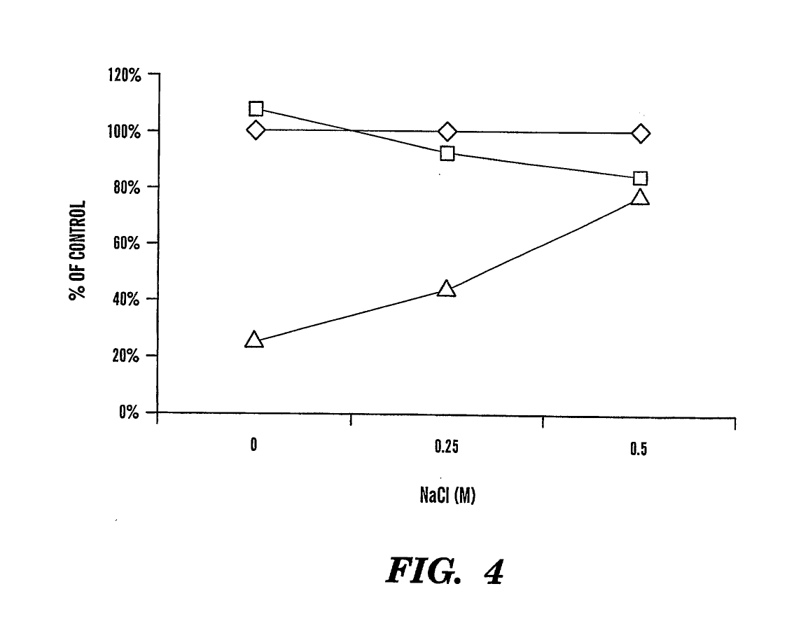

To determine if the mechanism of inhibition was based on an electrostatic interaction between heparin and the substrate that would effectively block acetylation sites, a binding assay was conducted. The substrate was incubated with sepharose or heparin-sepharose in the presence of increasing concentrations of sodium chloride Following centrifugation to pellet the beads, the resulting supernatant was assayed for protein content (FIG. 4). The resulting supernatant following incubation of the substrate with heparin-sepharose in the absence of NaCl had a ≈75% decrease in protein level compared to sepharose alone, indicating that the substrate bound heparin and consequently was not detected in the supernatant. However, increasing concentrations of NaCl resulted in increasing protein levels in the supernatant, suggesting that NaCl disrupted the interaction between the substrate and heparin-sepharose. A concentration of 0.5 M NaCl resulted in approximately 100% recovery of the substrate relative to pre-incubation with sepharose. Thus, heparin may inhibit HAT by interacting with the substrate and blocking acetylation sites.

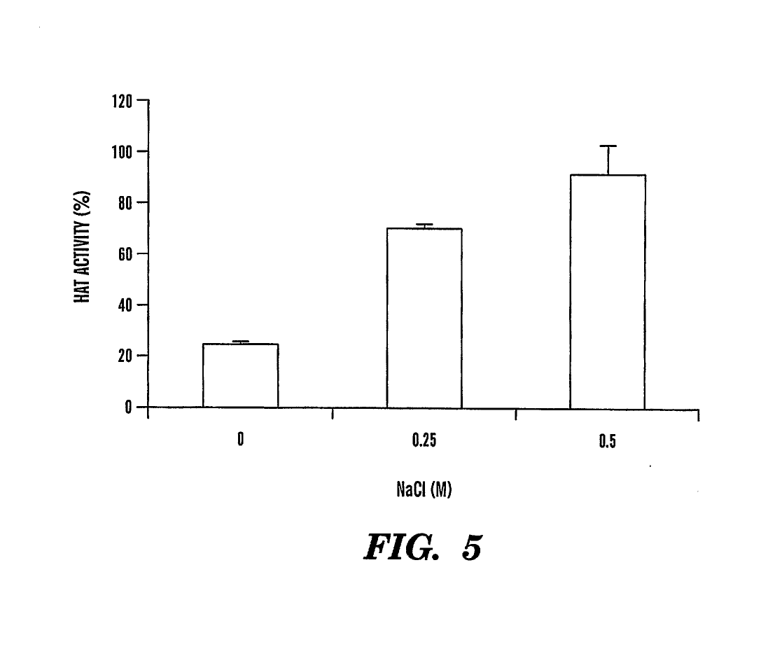

To examine the possibility that the mechanism of inhibition is based on an interaction between heparin and the enzyme, the enzyme was incubated with sepharose or heparin-sepharose in the presence of increasing concentrations of NaCl. Following centrifugation to pellet the beads, the resulting supernatant was included in the in vitro HAT assay along with the addition of the remaining assay components (i.e. substrate and [3H]-acetyl CoA) (FIG. 5). In the absence of NaCl, there was a low level of HAT activity following pre-incubation of the enzyme with heparin-sepharose relative to sepharose alone, suggesting that the enzyme bound to heparin and was trapped in the pellet following centrifugation. Thus, the enzyme was not available in the supernatant to catalyze the acetylation of substrate in the ensuing HAT assay. Along the same lines, the increase in HAT activity observed in the supernatant following pre-incubation with heparin-sepharose in the presence of high NaCl concentrations, suggests that NaCl disrupted the binding of enzyme to heparin. The binding of heparin to enzyme was similar to that observed with the substrate as 0.5 M NaCl was also sufficient to disrupt the putative HAT-heparin complexes as nearly full activity was recovered. Thus, the inhibition of HAT activity in vitro by heparin may involve electrostatic interactions with substrate and/or enzyme.

Cell Surface HSPG Ectodomains from CSF Inhibit Hat Activity In Vitro

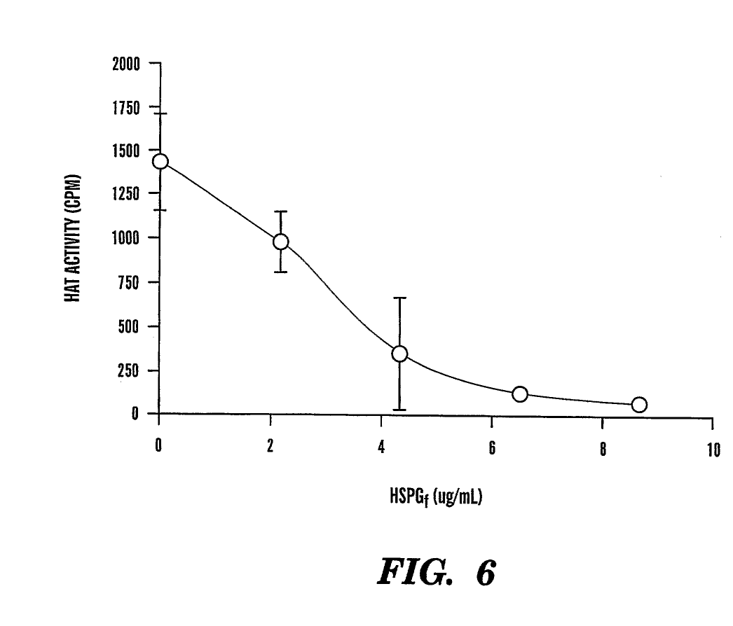

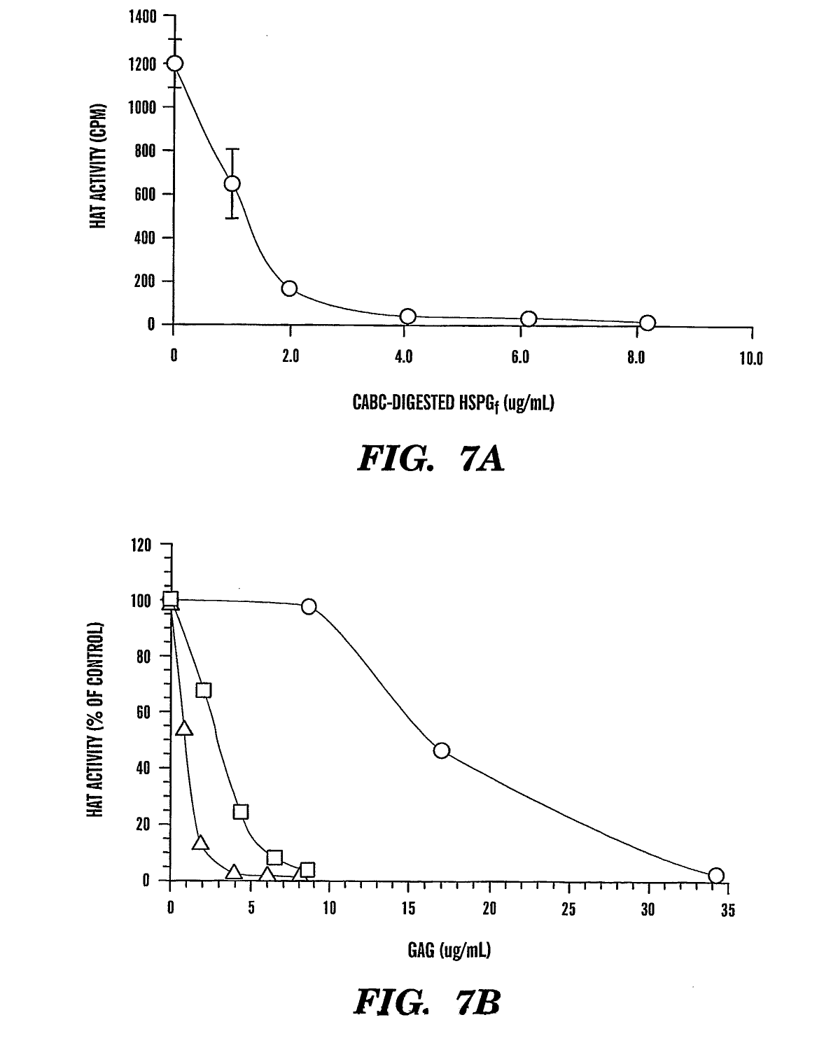

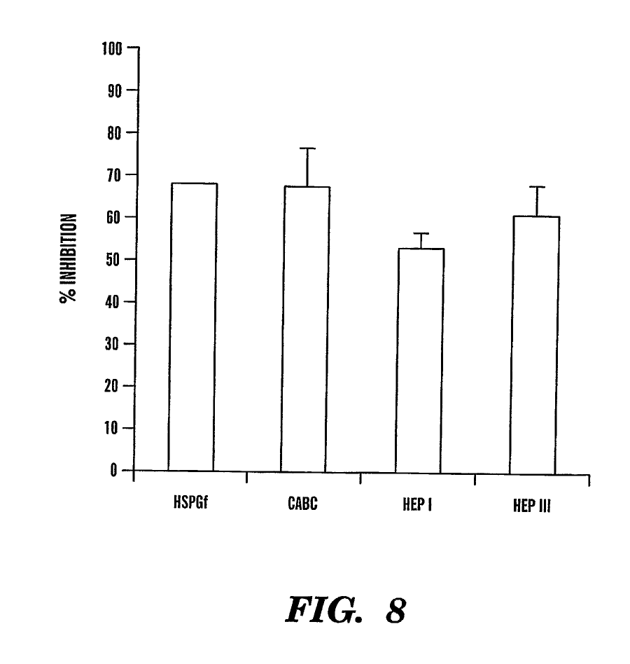

Although not wanting to be bound by theory, our underlying hypothesis concerning the function of nuclear HSPGs is that HS modulates transcription by regulating HAT activity. To examine the possibility that HSPG isolated from our cell system could inhibit HAT activity in vitro, we partially purified cell surface HSPG ectodomain fragments (HSPG_{f}) released by mild trypsin treatment using ion-exchange chromatography. This method has previously been shown to release syndecan ectodomains containing attached HS chains. HS-rich syndecan-4 ectodomains may be the HSPG component that localizes to the nucleus. Next, we conducted the in vitro HAT activity assay in the presence of HSPG_{f} (FIG. 6). Interestingly, HSPG_{f} decreased HAT activity in a dose-dependent manner, suggesting that HSPG from CSF inhibits HAT activity. Furthermore, the relative degree of inhibition was greater than that seen with heparin. The IC₅₀ of HSPG_{f} for HAT activity was calculated, by interpolating the concentration at which HAT activity was reduced to 50% relative to control, and was determined to be approximately 3 μg/mL. The IC₅₀ of heparin for HAT activity was calculated in the same way and determined to be approximately 17 μg/mL (see FIG. 3). Furthermore, a molar comparison strengthens this observation. Estimating that HSPG_{f} have a molecular weight of ˜150 kD, 3 μg/mL equates to approximately 20 nM. Similarly 17 μg/mL of heparin, with a molecular weight of ˜15 kD, equates to approximately 1.1 μM. Thus, based on a molar comparison, inhibition by HSPGf is approximately 55-fold greater than heparin, suggesting that HS may be a specific inhibitor of HAT activity in vitro. To further understand the importance of HS structure on the regulation of HAT activity, HSPGf was pre-digested with chondroitinase ABC (CABC) to degrade CS chains, which can sometimes be associated with syndecans (FIG. 7A). Interestingly, these CABC-digested HSPGf were even more potent than non-CABC-digested HSPGf, with an IC50 of approximately 1 μg/mL, suggesting that HS, and not CS, are the active components in mediating this inhibition. In fact, by comparing the dose responses of heparin, HSPGf, and CABC-digested HSPGf, it becomes obvious that there is an increase in the specific HAT inhibitory potential with HS compared to heparin, suggesting that HS contains specific structures that mediate this inhibition (FIG. 7B). HSPGf was also treated with other GAG chain lyases, such as heparinase I, and heparinase III, prior to inclusion in the in vitro HAT assay (FIG. 8). Pre-digestion with heparinases I and III resulted in slightly decreased HAT inhibition, suggesting that specific HS structure is important in dictating the specificity of inhibition. Since heparinase I targets highly sulfated regions of HS, while hep III targets regions of low sulfation (Ernst et al. 1995), these results indicate that HS structure can provide an additional level of specificity in the inhibition of HAT activity.

In addition to functioning as a nuclear shuttle for fibroblast growth factor 2 (Hsia et al., (2003)), nuclear HSPG modulates cellular activities by regulating HAT activity. We have shown that heparin decreases HAT activity in vitro. Although not wishing to be bound by theory, the mechanism of inhibition may involve the binding of heparin to both substrate and/or enzyme, thereby blocking both acetylation sites and catalytic activity. In addition, cell surface HSPG ectodomains isolated from CSF inhibited HAT in vitro, and this inhibition was even more pronounced than that of heparin, indicating that HS can specifically inhibit HAT. Moreover, various GAG lyase digestions of these CSF HSPG ectodomains revealed that the specific structure of HS appears to be an important determinant in the mechanism of this inhibition. The digestion of CS by CABC had a slightly greater effect on the inhibition of HAT activity, while the decrease in HAT inhibition due to digestion of HS by heparinase I was slightly different than that of hep III, indicating that HS sequence determines specificity of action.

The presence of HSPG and HS in the nucleus is a relatively novel concept; thus little is known about specific functions. The complex structure of HS chains allows potential interactions with a variety of molecules (David 1992; Turnbull et al. 2001; Shriver et al. 2002). Numerous combinations of acetylated and sulfated regions permit seemingly limitless possibilities for specific binding configurations. We teach that nuclear HSPG regulates histone acetyltransferase (HAT) activity by disrupting HAT-histone interactions, resulting in modification of gene transcription. We found that heparin, a molecule whose structure is similar to that of HS, inhibited HAT (specifically, p300) activity in vitro, and that HS can also inhibit HAT (FIG. 3). The mechanism of this inhibition seemed to involve binding of heparin to both the enzyme and histone substrate, as the presence of NaCl (<0.5 M) was sufficient to abrogate the inhibitory effect (FIG. 4 and FIG. 5). Utilizing the finding that trypsin releases syndecan ectodomains with intact HS chains (Rapraeger and Bernfield 1985), we were able to isolate and purify HSPG ectodomains (HSPGf) from CSF. Interestingly, HSPGf also inhibited in vitro HAT activity in a dose-dependent manner but even more potently than heparin (FIG. 6), showing that GAGs such as HSPG in CSF have the potential to inhibit HAT activity in vivo. We examined the contribution of HS to the inhibitory effects of HSPGf. We digested HSPGf with CABC, heparinase I (hep I), or heparinase III (hep III), and evaluated HAT activity in the presence of these digests (FIG. 7). Although the effect was minimal, CABC-digested HSPGf had a slightly greater inhibitory effect on HAT activity compared to un-digested HSPGf, which is consistent with HS, and not CS, being the active component in inhibiting HAT. Interestingly, hep I and hep III digestion of HSPGf resulted in slightly less HAT inhibition. Thus, the complex sequence arrangement of HS chains appear to be a critical parameter in determining the specificity of HAT inhibition.

### Example 2

### “Sequence” Specific Oligosaccharides Inhibit p300 Histone Acetyltransferase Activity In Vitro

Materials and methods

In Vitro HAT Activity Assay

HAT activity was assessed in vitro as outlined in FIG. 2. In a 1.7 mL microcentrifuge tube, 100 μL of substrate (biotinylated histone H4 peptide in 50 mM Tris, pH 7.4, 1 mM EDTA; from Pierce), 100 μL of a 5 mg/mL BSA solution (in H2O), 10.5 μL of 10×HAT buffer (500 mM Tris, pH 7.4, 10 mM EDTA), 20 U/mL of HAT enzyme (p300 HAT domain; from Upstate), and 1 μCi/mL of [³H] Acetyl-CoA (Amersham) were mixed together and the reaction was allowed to proceed at 30° C. for 1 hour. Following this incubation, 100 μL of a 1:1 slurry of immobilized streptavidin (Pierce) pre-equilibrated in 1×HAT buffer (50 mM Tris, pH 7.4, 1 mM EDTA) was added and the sample was incubated for 30 min at RT on a rotating platform. After centrifugation at 10,000 g for 4 min, the supernatant containing the excess reactants was removed and the pellet containing the acetylated substrate bound to the immobilized streptavidin was washed 3 times with 500 μL RIPA buffer (50 mM Tris, pH 7.4, 150 mM NaCl, 1 mM EDTA, 1% NP-40, 0.5% SDC, 0.1% SDS). After the final wash, the pellet was resuspended in 500 μL 1×HAT buffer, diluted in EcoLite, and counted in a scintillation counter. Because of sample volume restrictions, reaction volumes were scaled down in some experiments.

Inhibition of p300 Histone Acetyltransferase Activity by Specific Oligosaccharides.

Heparan sulfates and heparin are made up of repeating disaccharide units of varying structure (as many as 48 distinct disaccharides are proposed to exist) such that this class of molecules has the potential to contain an enormous amount of information. Indeed recent studies have begun to show that specific regions or “sequences” along the heparan sulfate chains allow for high affinity binding and modulation of a wide range of enzymes, hormones, and growth factors. Hence, small oligosaccharide chains of particular chemical sequence and composition are likely to show specificity for inhibitory activity of particular HAT enzymes.

The increased HAT inhibitory activity of the heparan sulfate proteoglycan fragments isolated from cells compared to heparin indicates that undersulfated regions might specifically inhibit HAT. This is based on the knowledge that heparan sulfate has a lower sulfate density when compared to heparin. Therefore we evaluated whether selectively de-sulfated heparin samples could retain inhibitory activity.

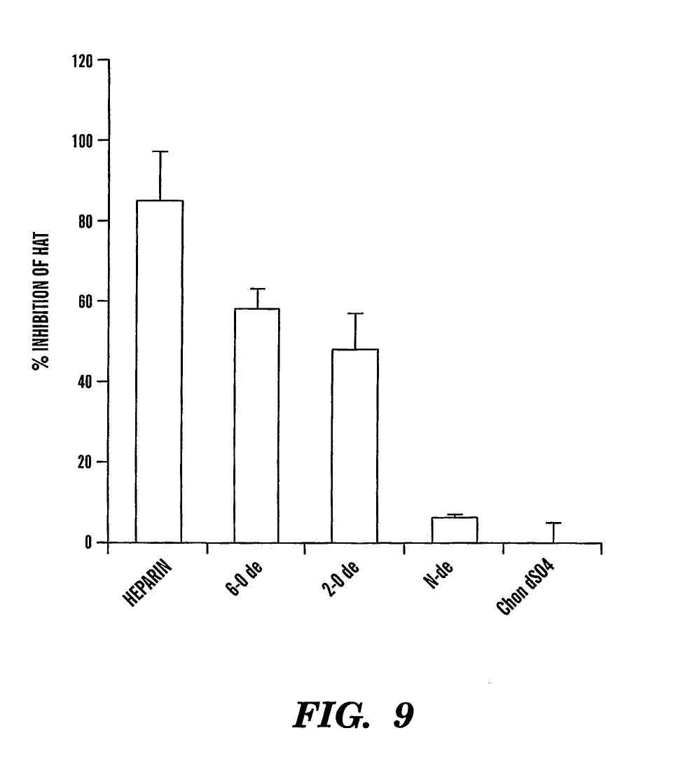

We used an In Vitro HAT assay to test the inhibitory activity of modified heparins. Heparin with the sulfates removed from the 6 position of the glucosamine (6-de), the 2 position of the uronic acid (2-de), or the N position of the glucosamine (N-de) were compared to each other, heparin and chondroitin sulfate (ChondSO4). The results are shown in FIG. 9. All samples were included in the assay at 10 ug/ml. 6-O and 2-O desulfated heparins retained HAT inhibitory activity indicating that neither O-sulfation on the 2-position of the uronic acid or the 6 position of the glucosamine residues are required for inhibitory activity. Thus, heparan sulfate-derived oligosaccharides can be developed which selectively inhibit HAT and not other proteins (e.g. non-anticoagulant heparins could be used).

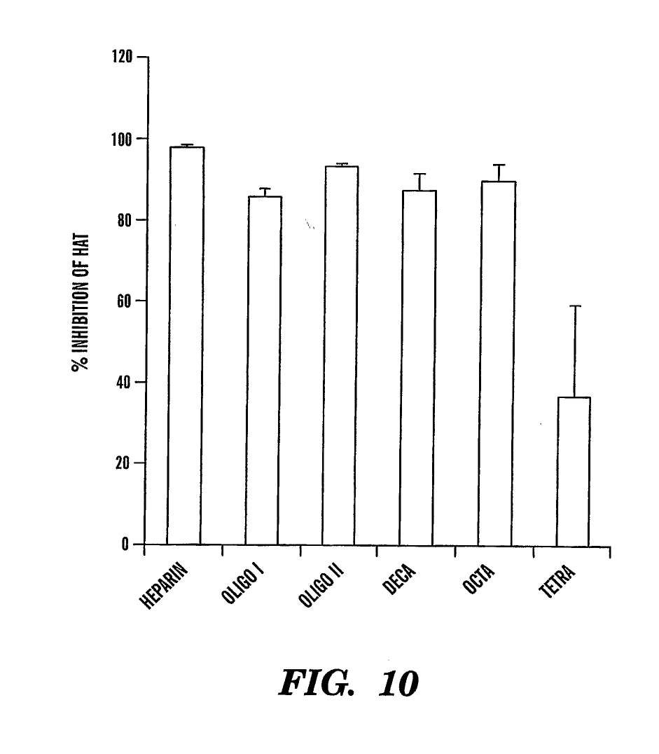

We also tested various sized oligosaccharide chains for HAT inhibitory activity. Various sized oligosaccharides derived from heparin were tested at 10 ug/ml for HAT inhibitory activity in an In Vitro HAT activity assay: Tetra (4 sugars), Octa (8 sugars), Deca (10 sugars), Oligo II (12-14 sugars), Oligo I (14-18 sugars). The results of which are shown in FIG. 10. Octosaccharides inhibited HAT activity as well as full length heparin, while oligosaccharides of 4 sugars did not.

### Example 3

### Inhibition of pCAF HAT Activity by Heparin, Modified Heparin, and Other Glycosaminoglycans In Vitro

### Materials and Methods

p300/CBP-associated factor (PCAF; histone acetyltransferase) was purchased from BIOMOL International (Plymouth Meeting, Pa.) and p300, HAT domain was purchased from Upstate (Lake Placid, N.Y.). For the HAT activity assays, heparin and the chemically modified heparin derivatives were purchased from Neoparin Inc. (San Leandro, Calif.). Chondroitin sulfate, D-glucosamine, glucutonic acid, N-acetylglucosamine, dextran and hyaluronic acid were purchased Sigma Chemical Company (St. Louis, Mo.) and the keratan sulfate and the Chondroitinase ABC were obtained from and Cape Cod Associates (Ijamsville, Md.). Porcine pancreatic elastase was purchased from Elastin Products (Owensville, Mich.). Antibodies to acetylated lysine and acetylated histone H3, the HAT assay substrates (biotinylated histone H3 and H4 peptides and core histones), and the salmon sperm DNA/Protein A Agarose were purchased from Upstate (Lake Placid, N.Y.). Horseradish peroxidase linked anti-rabbit IgG was purchased from Sigma Chemical Company and HRP-linked anti-mouse IgG and the ECL western blotting reagents were purchased from Amersham Biosciences (Piscataway, N.J.). Protran nitrocellulose for the vacuum filtration HAT assays was obtained Schleicher & Schuell (Keene, N.H.) and the Immobilon-P for western blotting analyses and the Amicon filters were obtained from Millipore Corporation (Bedford, Mass.). All chemicals and buffers for SDS PAGE and the protein assay reagent were obtained from Bio-Rad (Hercules, Calif.). The immobilized steptavidin was obtained from Pierce (Rockford, Ill.). The [³H]acetyl CoA and [³⁵S] Sulfate were obtained from Perkin Elmer (Boston, Mass.). All other chemicals were reagent grade products obtained from commercial sources.

### In Vitro HAT Assays

Heparin-mediated inhibition of HAT activity was determined using two independent methods. The first assay method used a modified protocol to measure the ability of pCAF or p300 to acetylate a synthetic, biotinylated peptide of histone H3 or H4 in the absence and presence of heparin or its derivatives. Commercially available pCAF or p300 HAT domain were added to an iced reactions mixture containing 3 μg biotinylated Histone H3 or H4 peptide, 50 mM tris pH 7.4, 1 mM EDTA with and without the indicated concentrations of heparin. 0.15 μCi [³H]acetyl-CoA was added to initiate the reaction and the samples were incubated for 30 minutes at 30° C. 100 μl prewashed, ImmunoPure Immobilized Streptavidin slurry was added to the reaction mixtures and the samples were incubated at room temperature for 1 hour with gentle agitation. The beads were centrifuged at 10,000 g for 4 minutes and the supernatants were discarded. The beads were washed 3 times with RIPA Buffer (50 mM tris pH 7.4, 150 mM sodium chloride, 1 mM EDTA, 1% NP-40, 0.5% deoxycholic acid, 0.1% SDS) prior to solubilization with 1N sodium hydroxide for 30 minutes at room temperature. Solubilized samples were processed for liquid scintillation counting. The second method for measuring heparin-mediated HAT inhibition utilized a modified filter binding assay (Sun, J. M., Spencer, V. A., Chen, H. Y., Li, L. and Davie, J. R. (2003) Methods 31, 12-23). 10 μg core histones were incubated on ice in buffer containing 50 mM tris pH 8.0, 1 mM DTT and 10% glycerol in the absence and presence of heparin or other GAG molecules. pCAF or p300 HAT domain was added to the reaction prior to the addition of 0.5 [Ci [³H]acetyl CoA to initiate the reaction. The reactions were incubated for 30 minutes at 30° C. 35 μl aliquots of the reaction mixture were spotted into wells of a dot blot apparatus and the samples were filtered through a Protran nitrocellulose membrane under vacuum to remove unincorporated acetyl CoA. The wells were washed 3 times under vacuum with 50 mM tris buffer pH 7.6. The nitrocellulose filter was removed from the blotter and was washed 3 additional times with tris buffer. The filter was allowed to air dry and the filters were processed and counted using liquid scintillation methods.

### Cell Culture

Primary cultures of pulmonary fibroblasts were isolated from the lungs of neonatal rats using established protocols (Foster, J. A., et al., (1990) Pulmonary fibroblasts: an in vitro model of emphysema. Regulation of elastin gene expression, J Biol Chem 265, 15544-9). The cells were maintained in Dulbecco's Minimal Essential Medium supplemented with 5% fetal bovine serum, 0.1 mM non-essential amino acid solution, 100 U/ml penicillin and 100 μg/ml streptomycin. The cells were used in second passage for all experiments. Cell number determination was made based upon assay of cellular acid phosphatase levels using a previously established method (Connolly, D. T., et al., (1986) Anal. Biochem. 152, 136-140).

### Generation and Purification of Elastase-Released Proteoglycans

Pulmonary fibroblasts were placed into second passage and were maintained for 10 days prior to elastase treatment. The cells were treated with 2.5 μg/ml porcine pancreatic elastase for 15 minutes at 37° C. The elastase supernatants were collected, inhibited with 1 mM diisopropyl fluorophosphate (DFP) and were stored at −80° C. prior to purification. Elastase released proteoglycans were purified using anion exchange chromatography using a modified protocol of Brown et al. (2002). The elastase digests were diluted with buffer containing 100 mM sodium acetate pH 6.0, 600 mM sodium chloride, 20 mM EDTA and 40% propylene glycol and were applied to a Q-sepharose column preequilibrated with buffer containing 50 mM sodium acetate, 300 mM sodium chloride, 10 mM EDTA and 20% propylene glycol (Q1 Buffer). The column was washed to baseline with Q1 Buffer and was washed with low salt buffer (50 mM sodium acetate pH 6.0, 300 mM sodium chloride). The proteoglycans were eluted with high salt buffer containing 50 mM sodium acetate pH 6.0 and 1.5M sodium chloride and the GAG-containing fractions were collected. The fractions were assayed for GAG content using the DMB assay (Farndale, R. W., Buttle, D. J. and Barrett, A. J. (1986) Biochimica et Biophysica Acta 883, 173-177 and for protein content using the Bio Rad protein assay). GAG containing fractions were pooled and were desalted/concentrated through and Amicon PL 10 filters and were exchanged into phosphate buffered saline (PBS) to generate the purified PG fraction (PG). The elastase-released heparan sulfate proteoglycan fragments (HSPGf) were produced upon treatment of the PG fraction with 10 mU/ml chondroitinase ABC for 6 hours at 37° C. and repurification using Q-sepharose based anion exchange methods (Brown, C. T., et al., (2002) Protein Expr Purif 25, 389-99; Brown, C. T., et al., (1999), J Biol Chem 274, 7111-9). Free GAG chains (B-PG) were generated by treating the purified PG fraction with 2M sodium borohydride in 0.1N sodium hydroxide for 16 hours at 37° C. using previously described methods (Forsten, K. E., et al., (1997), J. Cell. Physiol. 172, 209-220) with subsequent repurification of the free GAG chains using Q-sepharose anion exchange chromatography. Purified proteoglycan fractions were assayed for GAG and protein content, aliquotted and stored at −80° C.

### Nuclear Fractionation of Neonatal Rat Pulmonary Fibroblasts

Pulmonary fibroblasts were maintained for the indicated times and treatment conditions prior to nuclear fractionation using established protocols (Hsia, E., et al., (2003, J Cell Biochem 88, 1214-25; Sperinde, G. V. and Nugent, M. A. (1998), Biochemistry 37, 13153-13164; Sperinde, G. V. and Nugent, M. A. (2000), Biochemistry 39, 3788-3796). The cells were collected by trypsinization and 10% fetal bovine serum was added to each plate once cell lifting had occurred. The cells were collected, pooled and maintained for a minimum of 5 minutes at 37° C. to ensure trypsin inactivation. The cells were centrifuged at 800 g for 5 minutes at 4° C. and the supernatant was retained as the cell associated fi-action. The cell pellets were washed once with HB buffer containing 10 mM HEPES pH 7.9, 10 mM potassium chloride, 0.1 mM EDTA, 0.1 mM EGTA, 1 mM DTT and 0.5 mM PMSF. The cells were resuspended in 1 ml HB buffer and were incubated on ice at 4° C. for 20 minutes prior to the addition of 0.6% NP-40, votexing for 10 seconds and centrifugation at 12,000 g for 2 minutes at 4° C. The supernatants were retained and stored at −80° C. as the cytosolic fractions and the cell pellets were washed one additional time with HB buffer containing 0.6% NP-40. The resulting cell pellets were resuspended in DR buffer containing 20 mM HEPES pH 7.9, 420 mM potassium chloride, 1.5 mM magnesium chloride, 0.2 mM EDTA and 20% glycerol and were incubated for 30 minutes on ice at 4° C. The cells were vortexed and centrifuged for 2 minutes at 12,000 g. The resulting supernatants were collected as the nuclear fractions. Cross contamination of the cytosolic and nuclear fractions was assess by assaying all fractions for acid phosphatase activity (Connolly, D. T., et al., (1986), Anal. Biochem. 152, 136-140; Sperinde, G. V. and Nugent, M. A. (1998), Biochemistry 37, 13153-13164).

### ³⁵S Sulfate Radiolabeling and Analysis of ³⁵S Labeled Proteoglycans

Fibroblast cell cultures were seeded into second passage and were maintained overnight. The cells were metabolically radiolabeled with media supplemented with 75 μCi/ml ³⁵S-Sulfate for the indicated times in culture prior to cellular fractionation as described above. The cellular fractions were kept at −20° C. prior to filtration. The ³⁵S-labeled proteoglycan content of all cellular fractions was quantitated by cationic nylon vacuum filtration methods and the amount of ³⁵S-labeled heparan sulfate was determined by nitrous acid cleavage methods (Rapraeger, A. and Yeaman, C. (1989), Analytical Biochemistry 179, 361-365).

### Immunoprecipitation of Acetylated Histone H3

Aliquots of soluble nuclear proteins were diluted 1:5 with buffer containing 0.01% SDS, 1.1% Triton X-100, 1.2 mM EDTA, 16.7 mM Tris pH 8.1 and 150 mM sodium chloride. Salmon sperm DNA/Protein Agarose was added and the samples were precleared with gentle agitation for 1 hour at 4° C. The samples were centrifuged at 1000 g for 1 minute at 4° C. The supernatants were collected and incubated overnight at 4° C. with 20 ug anti-acetylated histone H3 antibody. 60 ul Salmon sperm DNA/Protein Agarose was added to each sample that was then incubated at 4° C. for 1 hour with gentle agitation. The samples were centrifuged at 1000 g at 4° C. The supernatant was removed and the resin was washed once with buffer containing 0.1% SDS, 1% Triton X-100, 2 mM EDTA, 20 mM tris pH 8.1 and 150 mM sodium chloride. The resin was equilibrated in the buffer for 5 minutes prior to centrifugation at 1000 g for 1 minute at 4° C. The supernatant was discarded and the resin was washed under the same conditions initially with buffer containing 0.1% SDS, 1% Triton X-100, 2 mM EDTA, 20 mM tris pH 8.1, 500 mM sodium chloride followed by a second wash with buffer containing 0.25M lithium chloride, 1% Triton X-100, 1% sodium deoxycholate, 1 mM EDTA and 10 mM tris pH 8.1. The resin was washed 2 additional times with buffer containing 10 mM tris pH 8.1 and 1 mM EDTA prior to solubilization with Laemelli Reducing Sample Buffer. The samples were stored at −20° C. prior to boiling for 10 minutes, separation on 17% SDS PAGE gels and electrotransfer to Immobilon membranes.

### Western Blot Analysis

Immobilon membranes were blocked for 1 hour at room temperature with blocking buffer containing 3% milk in tris buffered saline containing 0.1% tween-20 (TBST). The blots were rinsed twice with TBST prior to incubation with the primary antibody solutions. The blots were incubated with the appropriate antibody dilution for 1 hour at room temperature or overnight at 4° C. The blots were washed with TBST prior to incubation with the appropriate HRP-linked IgG for 1 hour at room temperature. The blots were washed with TBST prior to chemiluminescence exposure.

Glycosaminoglycans, Heparin and Modified Heparin are Inhibitors of p300 and PCAF HAT Activity.

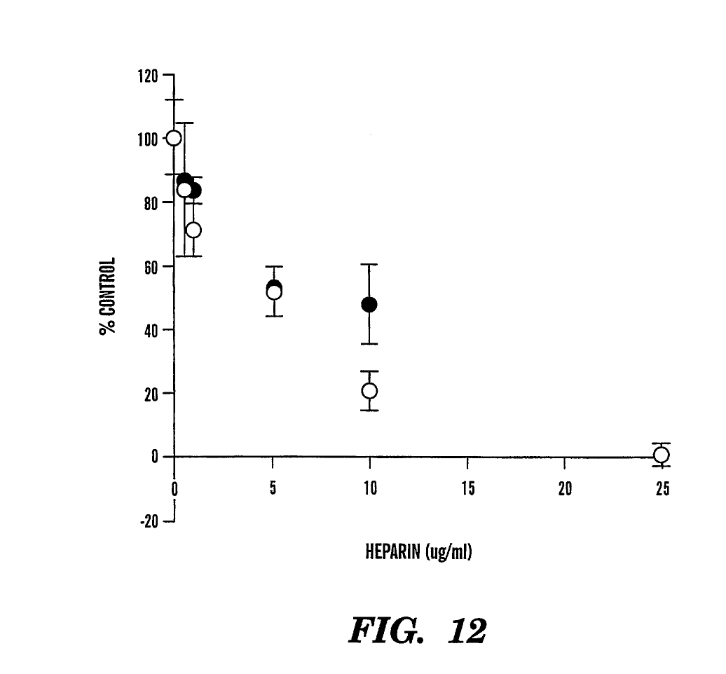

To determine if heparin can inhibit histone acetyltransferase activity toward intact histones, the acetylation of core histones was measured with two separate HAT enzymes, p300 and PCAF in the presence of various concentrations of porcine mucosa heparin (FIG. 12). Heparin was a potent inhibitor of both p300 and PCAF in this assay system with 50% inhibition (IC50) being observed with ˜5 and 7 μg/ml heparin for p300 and PCAF respectively.

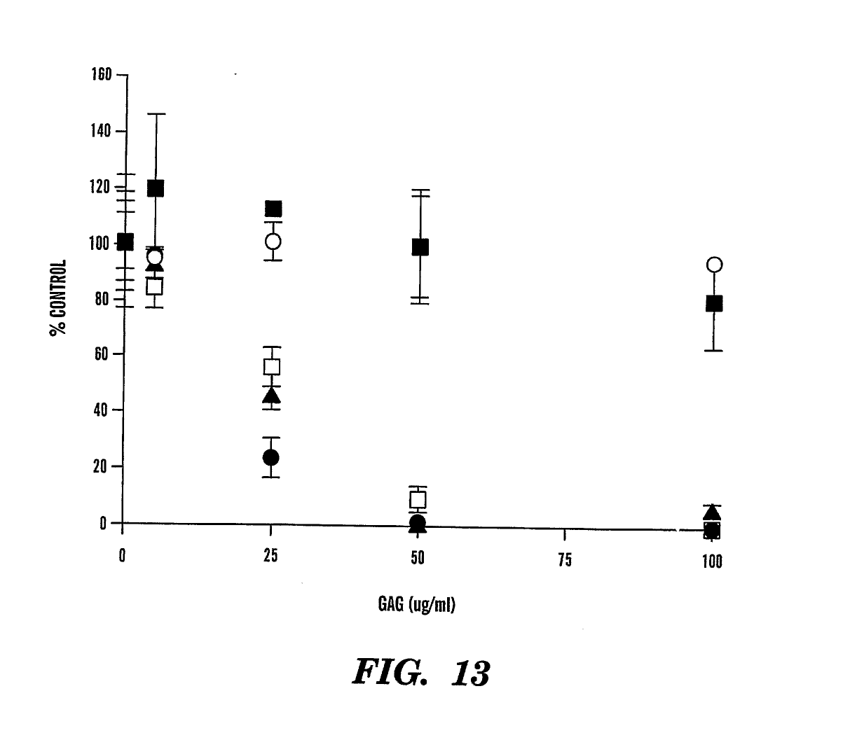

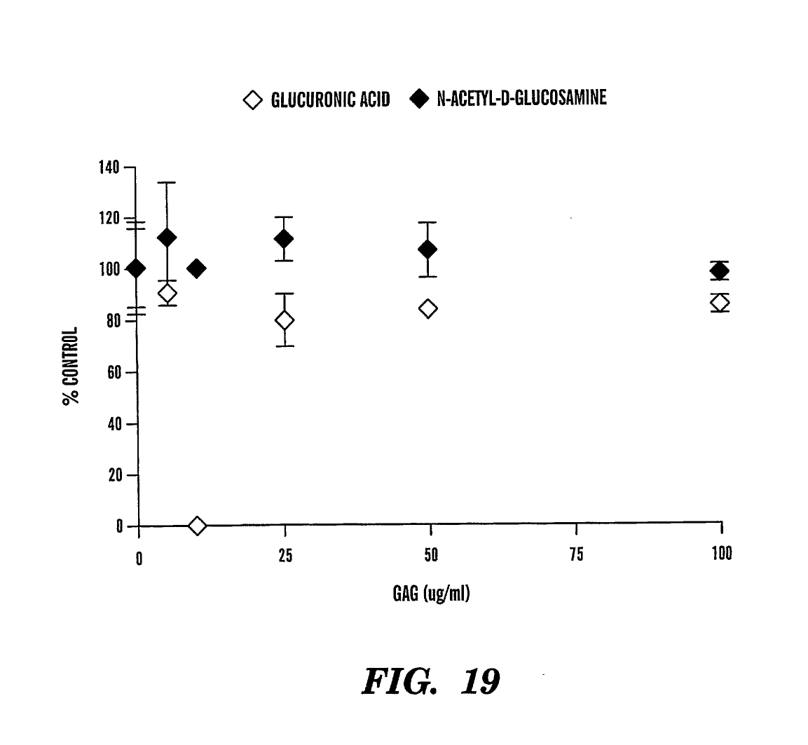

To determine if the inhibition of HAT activity was a general property of the chemical composition of heparin we evaluated the inhibitory activity of a series of related compounds including the saccharide building blocks of heparin: glucuronic acid, glucosamine, and N-acetyl glucosamine, as well as other polysaccharides: chondroitin sulfate, keratan sulfate, hyaluronic acid, and dextran. While none of the monosaccharides showed any significant inhibitory activity over the range of concentrations tested, chondroitin sulfate (CS), keratan sulfate (KS) and hyaluronic acid (HA) showed inhibition (FIG. 13, FIG. 19, and data not shown). Dextran polysaccharide did not show any inhibitory activity indicating that this activity is not a property of all polysaccharides. Moreover, none of the GAGs tested were as effective as heparin, and, consistent with a requirement for sulfation residues for full activity, the unsulfated GAG, HA, was the least effective.

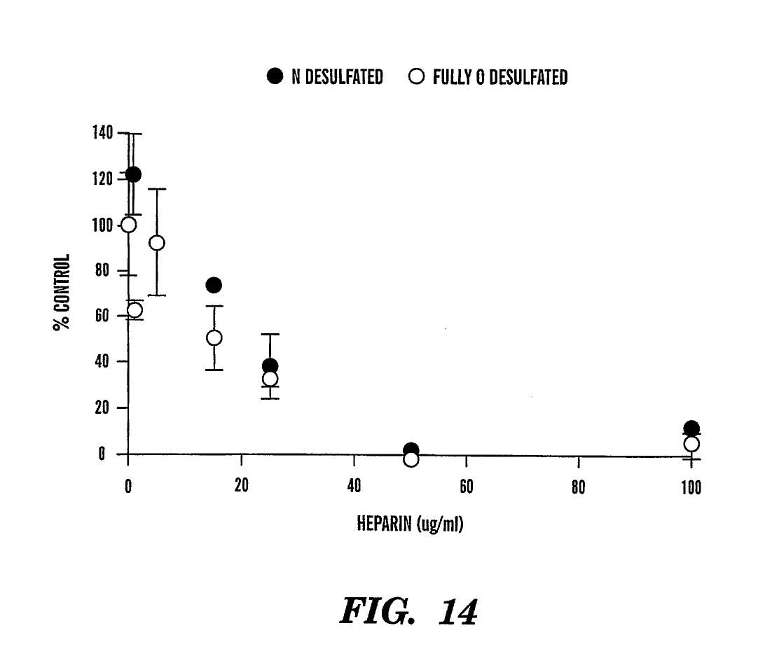

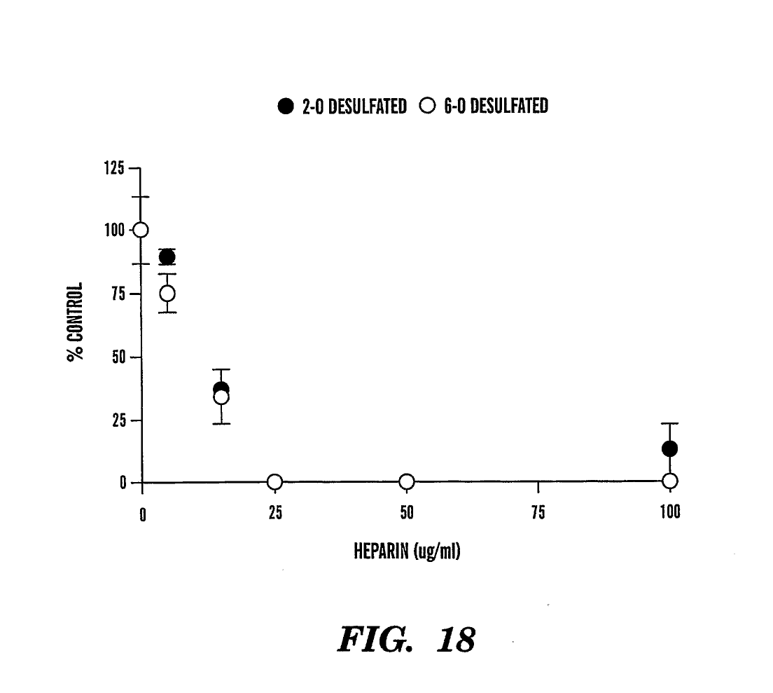

Heparin selectively de-sulfated at the 2-O position of the uronic acid or the 6-O position of the glucosamine showed reduced activity when compared to heparin, while removal of the sulfate from the N-group on the glucosamine nearly eliminated HAT (p300) inhibition with the H4 peptide (FIG. 9). Thus, we analyzed the effects of N-desulfated and O-desulfated (both 2-O and 6-O removed) heparin at a range of concentrations with PCAF and core histones (FIG. 14). Both desulfated heparins inhibited PCAF activity (IC50˜20 μg/ml); however, higher doses of the desulfated heparins were required to achieve similar levels of inhibition as that observed with heparin. This observation is consistent with the relative activities of other GAGs, as the undersulfated and unsulfated GAGs (i.e. CS and HA) produced a similar inhibition profile as that observed with the desulfated heparin samples. Thus, full heparin-mediated HAT inhibition requires sulfation on N and O groups. While sulfation on N groups appears to be a requirement, the selective removal of only 2-O or 6-O sulfation did not result in significant loss of function indicating that sulfation at either of these two O-positions is the minimum requirement for activity (FIG. 18).

Elastase Generated Proteoglycans Inhibit pCAF HAT Activity

Since excessive HAT activity has been implicated in the progression of chronic obstructive pulmonary disease (COPD), we decided to investigate the activity of heparan sulfate proteoglycan (HSPG) in the pulmonary system. COPD, and particularly emphysema, has been linked to excessive elastase degradation through the action of neutrophil and macrophage produced proteases. In normal circumstances the action of elastase is likely coupled to repair processes. We have noted that elastase releases proteoglycans (PGs; including HSPGs) from pulmonary cells and lung tissue through partial degradation of the PG core proteins. We tested the hypothesis that the elastase-generated soluble HSPG fragments feed-back to regulate important cell functions such as HAT activity that they may play critical roles in repair of damaged tissue. We also tested whether endogenous, undigested, HSPGs cycle normally to the nucleus within these cells to control HAT activity.

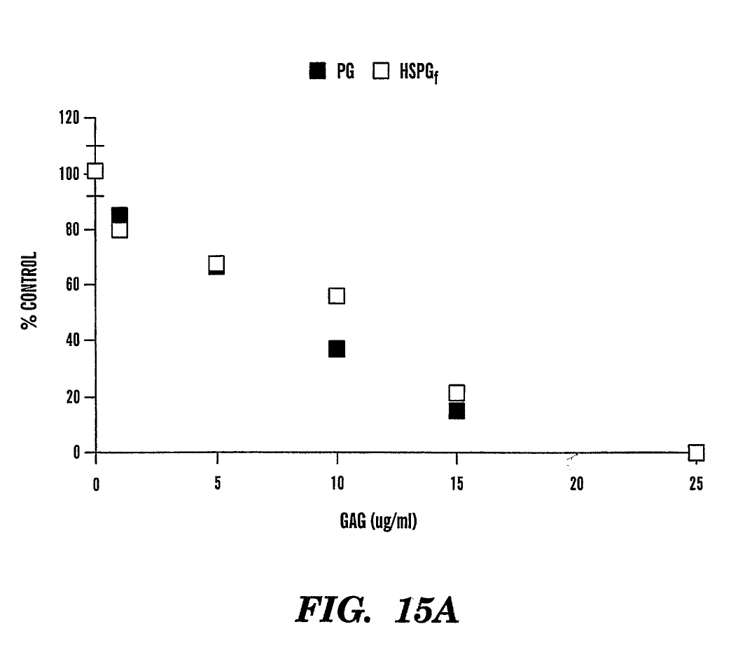

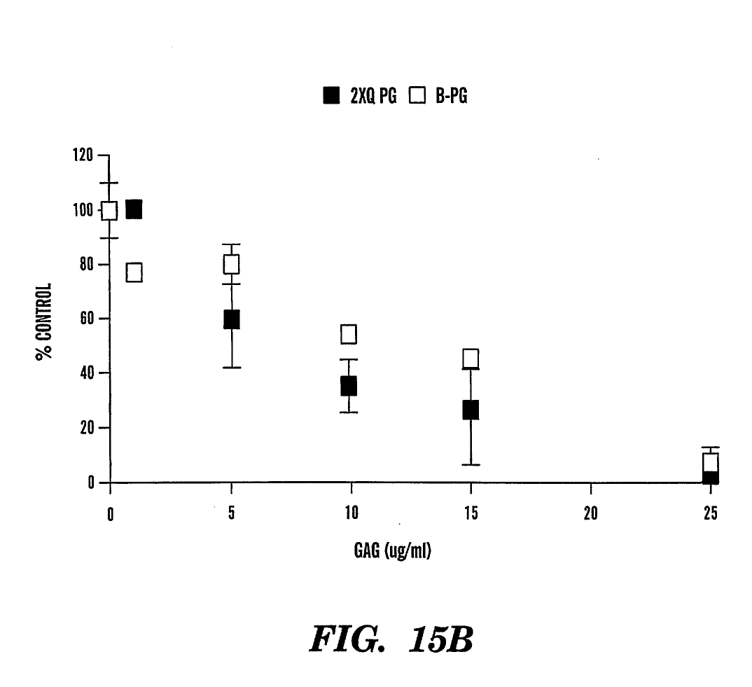

We isolated PG fragments from primary pulmonary fibroblast by subjecting them to mild elastase digestion. The soluble PG fragments were purified by ion exchange chromatography and included in in vitro PCAF assays (FIG. 15). Isolated PG fragments showed significant dose dependent inhibition of PCAF with an IC50 ˜7 μg/ml. This activity was a reflection of the HS present, as complete digestion of CS with chondroitinase ABC and re-isolation of the HSPG fragments did not result in any significant loss of activity. To further verify that the PCAF inhibitory activity was the result of the HS chains and did not require PG core proteins, we released the GAG chains from the core protein by beta elimination in sodium borohydride (B-PG). These isolated GAG chains were repurified by ion exchange and compared to un-treated PG that was also subjected to re-purification (FIG. 15B). The isolated GAG chains showed similar activity as the intact PG fragments. Together these results demonstrate that elastase released PGs can inhibit HAT activity via the action of HS chains.

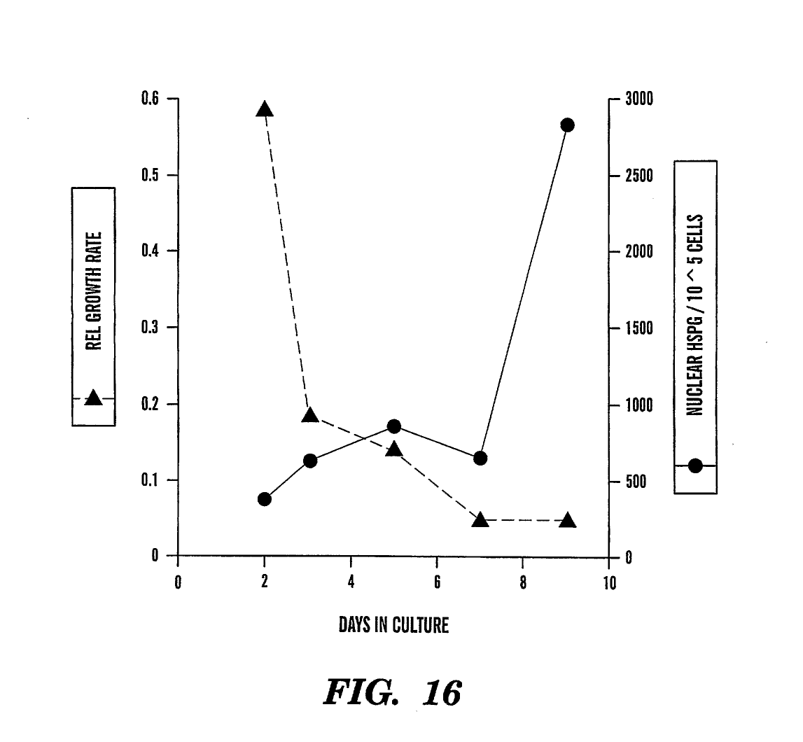

In an attempt to determine if HSPG within these cells are playing important roles as regulators of nuclear activity, we conducted an analysis of cell growth rate and nuclear HSPG levels at various times in culture. We have characterized this pulmonary cell system extensively in the past and have shown that the cells undergo a phenotypic change with time in culture. At early times, 1-4 days, the cells grow rapidly and produce very little extracellular matrix (specifically elastin). After 7-9 days, the cells become quiescent and begin to produce significant amounts of extracellular matrix. We have used this system to investigate the components involved in the transition of these cells from the pre- to post-elastogenic state. We tested whether nuclear HSPG inhibits HAT activity and stimulates the exit of these cells from the cell cycle. To evaluate this possibility, we biosynthetically labeled the HSPG in these cells with ³⁵SO₄ and, at various times in culture, we isolated nuclei, extracted the proteoglycans and quantitated the levels of HSPG by Zetaprobe analysis. We noted relatively little nuclear HSPG in these cells at the early times when the cells were rapidly growing; however, as the cells reached a quiescent state (day 9) we noted a dramatic increase in nuclear HSPG levels (FIG. 16).

### Heparin Inhibits Histone H3 Acetylation in Pulmonary and Smooth Muscle Cells.

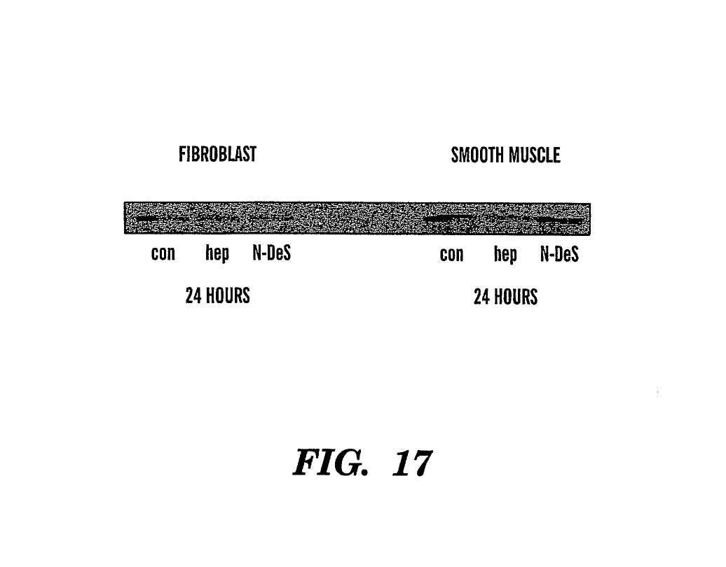

To test whether nuclear HSPG participate in modulating HAT activity endogenously we added heparin and the less active N-desulfated heparin to these cells (10 μg/ml, for 24 h) and analyzed the level of histone H3 acetylation by western blot. FIG. 17 shows that heparin treatment reduced histone H3 actetylation slightly. We also treated another cell type, aortic smooth muscle cells, with heparin to determine if this response was general or specific to the pulmonary cells. We observed a significant reduction of histone H3 acetylation in smooth muscle cells treated with heparin, but not in cells treated with N-desulfated heparin.

All references described herein are incorporated herein by reference.

### REFERENCES

Aisen, P. (1988). “Iron metabolism in isolated liver cells.” Annals of the New York Academy of Sciences 526: 93-100.
Amalric F, G. Bouche, H. Bonnet, P. Brethenou, A. M. Roman, I. Truchet, N. Quarto (1994). “Fibroblast growth factor-2 (FGF-2) in the nucleus: translocation process and targets.” Biochemical Pharmacology 47(1): 111-115.
Arroyo-Yanguas, Y., F. Cheng, A. Isaksson, L. A. Fransson, A. Malmstrom, G. Westergren-Thorsson (1997). “Binding, internalization, and degradation of antiproliferative heparan sulfate by human embryonic lung fibroblasts.” Journal of Cellular Biochemistry 64(4): 595-604.
Asthagiri, A. R., C. M. Nelson, A. F. Horwitz, D. A. Lauffenburger (1999). “Quantitative relationship among integrin-ligand binding, adhesion, and signaling via focal adhesion kinase and extracellular signal-regulated kinase 2.” Journal of Biological Chemistry 274(38): 27119-27.
Bailly K, S. F., D. Leroy, F. Amalric, G. Bouche (2000). “Uncoupling of cell proliferation and differentiation activities of basic fibroblast growth factor.” FASEB Journal 14: 333-344.
Barnes, P. J. (2003) Theophylline: new perspectives for an old drug, Am J Respir Crit. Care Med 167, 813-8
Barnes, P. J., Ito, K. and Adcock, I. M. (2004) Corticosteroid resistance in chronic obstructive pulmonary disease: inactivation of histone deacetylase, Lancet 363, 731-3.
Bass, M. D. and M. J. Humphries (2002). “Cytoplasmic interactions of syndecan-4 orchestrate adhesion receptor and growth factor receptor signalling.” Biochemical Journal 368 (Pt 1): 1-15.
Bickel, P. E., P. E. Scherer, J. E. Schnitzer, P. Oh, M. P. Lisanti, H. F. Lodish (1997). “Flotillin and epidermal surface antigen define a new family of caveolae-associated integral membrane proteins.” Journal of Biological Chemistry 272(21): 13793-802.
Bonnet, H., O. Filhol, I. Truchet, P. Brethenou, C. Cohet, F. Amalric, G. Bouche (1996). “Fibroblast growth factor-2 binds to the regulatory b subunit of CK2 activity toward nucleolin.” Journal of Biological Chemistry 271(40): 24781-24787.
Bredt, D. S. (2000). “Cell biology. Reeling CASK into the nucleus.” Nature 404(6775): 241-2.
Brooks, C. L. and W. Gu (2003). “Ubiquitination, phosphorylation and acetylation: the molecular basis for p53 regulation.” Current Opinion in Cell Biology 15(2): 164-71.
Brown, C. E., T. Lechner, L. Howe, J. L. Workman (2000). “The many HATs of transcription coactivators.” Trends in Biochemical Sciences 25(1): 15-9.
Brown, C. T., E. Applebaum, R. Banwatt, V. Trinkaus-Randall (1995). “Synthesis of stromal glycosaminoglycans in response to injury.” Journal of Cellular Biochemistry 59(1): 57-68.
Brown, C. T., P. Lin, M. T. Walsh, D. Gantz, M. A. Nugent, V. Trinkaus-Randall (2002). “Extraction and purification of decorin from corneal stroma retain structure and biological activity.” Protein Expression and Purification 25(3): 389-99.
Brown, D. A. and E. London (2000). “Structure and function of sphingolipid- and cholesterol-rich membrane rafts.” Journal of Biological Chemistry 275(23): 17221-4.
Brown, D. A. and J. K. Rose (1992). “Sorting of GPI-anchored proteins to glycolipid-enriched membrane subdomains during transport to the apical cell surface.” Cell 68(3): 533-44.
Brownell, J. E., J. Zhou, T. Ranalli, R. Kobayashi, D. G. Edmondson, S. Y. Roth, C. D. Allis (1996). “Tetrahymena histone acetyltransferase A: a homolog to yeast Gcn5p linking histone acetylation to gene activation.” Cell 84(6): 843-51.
Buczek-Thomas, J. A. and M. A. Nugent (1999). “Elastase-mediated release of heparan sulfate proteoglycans from pulmonary fibroblast cultures. A mechanism for basic fibroblast growth factor (bFGF) release and attenuation of bfgf binding following elastase-induced injury.” Journal of Biological Chemistry 274(35): 25167-72.
Byzova, T. V., C. K. Goldman, N. Pampori, K. A. Thomas, A. Bett, S. J. Shattil, E. F. Plow (2000). “A mechanism for modulation of cellular responses to VEGF: activation of the integrins.” Molecular Cell 6(4): 851-60.
Carey, D. J. (1997). “Syndecans: multifunctional cell-surface co-receptors.” Biochemical Journal 327 (Pt 1): 1-16.
Chan, H. M. and N. B. La Thangue (2001). “p300/CBP proteins: HATs for transcriptional bridges and scaffolds.” Journal of Cell Science 114 (Pt 13): 2363-73.
Cheng, F., P. Petersson, Y. Arroyo-Yanguas, G. Westergren-Thorsson (2001). “Differences in the uptake and nuclear localization of anti-proliferative heparan sulfate between human lung fibroblasts and human lung carcinoma cells.” Journal of Cellular Biochemistry 83(4): 597-606.
Clayton, A., J. Thomas, G. J. Thomas, M. Davies, R. Steadman. (2001). “Cell surface heparan sulfate proteoglycans control the response of renal interstitial fibroblasts to fibroblast growth factor-2.” Kidney International 59(6): 2084-94.
Cohen, A. R., D. F. Woods, S. M. Marfatia, Z. Walther, A. H. Chishti, J. M. Anderson, D. F. Wood (1998). “Human CASK/LIN-2 binds syndecan-2 and protein 4.1 and localizes to the basolateral membrane of epithelial cells.” Journal of Cell Biology 142(1): 129-38.
Cosio, B. G., Tsaprouni, L., Ito, K., Jazrawi, E., Adcock, I. M. and Barnes, P. J. (2004) Theophylline restores histone deacetylase activity and steroid responses in COPD macrophages, J Exp Med 200, 689-95
Couchman, J. R. and A. Woods (1999). “Syndecan-4 and integrins: combinatorial signaling in cell adhesion.” Journal of Cell Science 112 (Pt 20): 3415-20.
David, G. (1992). “Structural and functional diversity of the heparan sulfate proteoglycans.” Advances in Experimental Medicine and Biology 313: 69-78.
de Ruijter, A. J., A. H. van Gennip, H. N. Caron, S. Kemp, A. B. van Kuilenburg (2003). “Histone deacetylases (HDACs): characterization of the classical HDAC family.” Biochemical Journal 370 (Pt 3): 737-49.
Du Clos, T. W., M. A. Volzer, F. F. Hahn, R. Xiao, C. Mold, R. P. Searles (1999). “Chromatin clearance in C57B1/10 mice: interaction with heparan sulphate proteoglycans and receptors on Kupffer cells.” Clinical and Experimental Immunology 117(2): 403-11.
Dudas, J., G. Ramadori, T. Knittel, K. Newbauer, D. Raddatz, K. Egedy, I. Kovalszky. (2000). “Effect of heparin and liver heparan sulphate on interaction of HepG2-derived transcription factors and their cis-acting elements: altered potential of hepatocellular carcinoma heparan sulphate.” Biochemical Journal 350 Pt 1: 245-51.
Ernst, S., R. Langer, C. L. Cooney, R. Sasisekharan (1995). “Enzymatic degradation of glycosaminoglycans.” Critical Reviews in Biochemistry and Molecular Biology 30(5): 387-444.
Esko, J. D. and S. B. Selleck (2002). “ORDER OUT OF CHAOS: Assembly of Ligand Binding Sites in Heparan Sulfate.” Annual Review of Biochemistry 71: 435-71.
Fannon, M. and M. A. Nugent. (1996). “Basic fibroblast growth factor binds its receptors, is internalized, and stimulates DNA synthesis in Balb/c3T3 cells in the absence of heparan sulfate.” Journal of Biological Chemistry 271: 17949-17956.
Farndale, R. W., D. J. Buttle, A. J. Barrett (1986). “Improved quantitation and discrimination of sulphated glycosaminoglycans by use of dimethylmethylene blue.” Biochimica et Biophysica Acta 883(2): 173-7.
Fedarko, N. S, and H. E. Conrad (1986). “A unique heparan sulfate in the nuclei of hepatocytes: structural changes with the growth state of the cells.” Journal of Cell Biology 102(2): 587-99.
Fedarko, N. S., M. Ishihara, H. E. Conrad. (1989). “Control of cell division in hepatoma cells by exogenous heparan sulfate proteoglycan.” Journal of Cellular Physiology 139(2): 287-94.
Ferrara, N. (2002). “VEGF and the quest for tumour angiogenesis factors.” Nature Reviews Cancer 2(10): 795-803.
Ferrara, N., H. P. Gerber, J. LeCouter. (2003). “The biology of VEGF and its receptors.” Nature Medicine 9(6): 669-76.
Filla M S, P. Dam, A. C. Rapraeger (1998). “The cell surface proteoglycan syndecan-1 mediates fibroblast growth factor-2 binding and activity.” Journal of Cellular Physiology 174: 310-321.
Fini, M. E. (1999). “Keratocyte and fibroblast phenotypes in the repairing cornea.” Progress in Retinal and Eye Research 18(4): 529-51.
Fuki, I. V., M. E. Meyer, K. J. Williams. (2000). “Transmembrane and cytoplasmic domains of syndecan mediate a multi-step endocytic pathway involving detergent-insoluble membrane rafts.” Biochemical Journal 351 Pt 3: 607-12.
Funderburgh, J. L. and J. W. Chandler (1989). “Proteoglycans of rabbit corneas with nonperforating wounds.” Investigative Opthalmology & Visual Science 30(3): 435-42.
Gallo, R., C. Kim, R. Kokenyesi, N. S. Adzick, M. Bernfield (1996). “Syndecans-1 and -4 are induced during wound repair of neonatal but not fetal skin.” Journal of Investigative Dermatology 107(5): 676-83.
Gao, Y., M. Li, W. Chen, M. Simons (2000). “Synectin, syndecan-4 cytoplasmic domain binding PDZ protein, inhibits cell migration.” Journal of Cellular Physiology 184(3): 373-9.
Gengrinovitch, S., B. Berman, G. David, L. Witte, G. Neufeld, D. Ron (1999). “Glypican-1 is a VEGF165 binding proteoglycan that acts as an extracellular chaperone for VEGF165.” Journal of Biological Chemistry 274(16): 10816-22.
Giordano, A. and M. L. Avantaggiati (1999). “p300 and CBP: partners for life and death.” Journal of Cellular Physiology 181(2): 218-30.
Gleizes, P. E., J. Noaillac-Depeyre, M. A. Dupont, N. Gas. (1996). “Basic fibroblast growth factor (FGF-2) is addressed to caveolae after binding to the plasma membrane of BHK cells.” European Journal of Cell Biology 71(2): 144-53.
Goerges, A. L. and M. A. Nugent (2003). “Regulation of vascular endothelial growth factor binding and activity by extracellular pH.” Journal of Biological Chemistry 278(21): 19518-25.
Grant, P A & Berger, S L (1999): Semin Cell Dev Biol, 10:169177.
Gregory, P. D., K. Wagner, W. Horz (2001). “Histone acetylation and chromatin remodeling.” Experimental Cell Research 265(2): 195-202.
Grootjans, J. J., P. Zimmermann, G. Reekmans, A. Smets, G. Degeest, J. Durr, G. David (1997). “Syntenin, a PDZ protein that binds syndecan cytoplasmic domains.” Proceedings of the National Academy of Sciences USA 94: 13683-13688.
Gschwendt, M., H. J. Muller, K. Kielbassa, R. Zang, W. Kittstein, G. Rincke, F. Marks (1994). “Rottlerin, a novel protein kinase inhibitor.” Biochemical and Biophysical Research Communications 199(1): 93-8.
Gu, W. and R. G. Roeder (1997). “Activation of p53 sequence-specific DNA binding by acetylation of the p53 C-terminal domain.” Cell 90(4): 595-606.
Harrington E O, J. Loffler, P. R. Nelson, K. C. Kent, M. Simons, J. A. Ware (1997). “Enhancement of migration by protein kinase Ca and inhibition of proliferation and cell cycle progression by protein kinase Cd in capillary endothelial cells.” Journal of Biological Chemistry 272(11): 7390-7397.
Hassell, J. R., C. Cintron, C. Kublin, D. A. Newsome (1983). “Proteoglycan changes during restoration of transparency in corneal scars.” Archives of Biochemistry and Biophysics 222(2): 362-9.
Haystead, T. A., A. T. Sim, D. Carling, R. C. Honnor, Y. Tsukitani, P. Cohen, D. G. Hardie (1989). “Effects of the tumour promoter okadaic acid on intracellular protein phosphorylation and metabolism.” Nature 337(6202): 78-81.
Hileman, R. E., J. R. Fromm, J. M. Weiler, R. J. Linhardt (1998). “Glycosaminoglycan-protein interactions: definition of consensus sites in glycosaminoglycan binding proteins.” Bioessays 20(2): 156-67.
Hocking, D. C. and K. Kowalski (2002). “A cryptic fragment from fibronectin's III1 module localizes to lipid rafts and stimulates cell growth and contractility.” Journal of Cell Biology 158(1): 175-84.
Horowitz, A., M. Murakami, Y. Gao, M. Simons (1999). “Phosphatidylinositol-4,5-bisphosphate mediates the interaction of syndecan-4 with protein kinase C.” Biochemistry 38(48): 15871-7.
Horowitz, A. and M. Simons (1998a). “Phosphorylation of the cytoplasmic tail of syndecan-4 regulates activation of protein kinase Ca.” Journal of Biological Chemistry 273(40): 25548-25551.
Horowitz, A. and M. Simons (1998b). “Regulation of syndecan-4 phosphorylation in vivo.” Journal of Biological Chemistry 273(18): 10914-10918.
Horowitz, A., E. Tkachenko, M. Simons (2002). “Fibroblast growth factor-specific modulation of cellular response by syndecan-4.” Journal of Cell Biology 157(4): 715-25.
Hsia, E., T. P. Richardson, M. A. Nugent (2003). “Nuclear localization of basic fibroblast growth factor is mediated by heparan sulfate proteoglycans through protein kinase C signaling.” Journal of Cellular Biochemistry 88(6): 1214-25.
Hsuch, Y. P., T. F. Wang, F. C. Yang, M. Sheng (2000). “Nuclear translocation and transcription regulation by the membrane-associated guanylate kinase CASK/LIN-2.” Nature 404(6775): 298-302.
Hsuch, Y. P., F. C. Yang, V. Kharazia, S, Naisbitt, A. R. Cohen, R. J. Weinberg, M. Sheng (1998). “Direct interaction of CASK/LIN-2 and syndecan heparan sulfate proteoglycan and their overlapping distribution in neuronal synapses.” Journal of Cell Biology 142(1): 139-51.
Hutchings, H., N. Ortega, J. Plouet (2003). “Extracellular matrix-bound vascular endothelial growth factor promotes endothelial cell adhesion, migration, and survival through integrin ligation.” FASEB Journal 17(11): 1520-2.
Immergluck, L. C., M. S. Domowicz, M. B. Schwartz, B. C. Herold (1998). “Viral and cellular requirements for entry of herpes simplex virus type 1 into primary neuronal cells.” Journal of General Virology 79 (Pt 3): 549-59.
Ingber, D. E. (1990). “Fibronectin controls capillary endothelial cell growth by modulating cell shape.” Proceedings of the National Academy of Sciences USA 87(9): 3579-83.
Ishihara, M., N. S. Fedarko, H. E. Conrad (1986). “Transport of heparan sulfate into the nuclei of hepatocytes.” Journal of Biological Chemistry 261(29): 13575-80.
K., Ito, M., Elliott, W. M., Cosio, B., Caramori, G., Kon, O. M., Barczyk, A., Hayashi, S., Adcock, I. M., Hogg, J. C. and Barnes, P. J. (2005) Decreased histone deacetylase activity in chronic obstructive pulmonary disease, N Engl J Med 352, 1967-76
Jacobson, R. H., A. G. Ladurner, D. S. King, R. Tijan (2000). “Structure and function of a human TAFII250 double bromodomain module.” Science 288(5470): 1422-5.
Jans, D. A. and G. Hassan (1998). “Nuclear targeting by growth factors, cytokines, and their receptors: a role in signaling?” Bioessays 20(5): 400-11.
Jester, J. V., P. A. Barry, G. J. Lind, W. M. Petroll, R. Garana, H. D. Cavanagh (1994). “Corneal keratocytes: in situ and in vitro organization of cytoskeletal contractile proteins.” Investigative Opthalmology & Visual Science 35(2): 730-43.
Joy, A., J. Moffett, K. Neary, E. Mordechai, E. K. Stacliowiak, S. Coons, R. Rankin-Shapiro, R. Z. Florkiewicz, M. K. Stachowiak (1997). “Nuclear accumulation of FGF-2 is associated with proliferation of human astrocytes and glioma cells.” Oncogene 14(2): 171-83.
Juan, L. J., W. J. Shia, M. H. Chen, W. M. Yang, E. Seto, Y. S. Lin, C. W. Wu (2000). “Histone deacetylases specifically down-regulate p53-dependent gene activation.” Journal of Biological Chemistry 275(27): 20436-43.
Keresztes, M. and J. Boonstra (1999). “Import(ance) of growth factors in (to) the nucleus.” J Cell Biol 145(3): 421-4.
Kim, Y., H. Park, Y. Lim, I. Han, H. J. Kwon, A. Woods, E. S. Oh (2003). “Decreased syndecan-2 expression correlates with trichostatin-A induced-morphological changes and reduced tumorigenic activity in colon carcinoma cells.” Oncogene 22(6): 826-30.
Kouzarides, T. (2000). “Acetylation: a regulatory modification to rival phosphorylation?” EMBO Journal 19(6): 1176-9.
Kovalszky I, J. Dudas, J. Olah-Nagy, G. Pogany, J. Tovary, J. Timar, L. Kopper, A.
Jeney, R. Iozzo (1998). “Inhibition of DNA topoisomerase I activity by heparan sulfate and modulation by basic fibroblast growth factor.” Molecular and Cellular Biochemistry 183: 11-23.
Krieger, M., P. Reddy, K. Kozarsky, D. Kingsley, L. Hobbie, M. Penman (1989). “Analysis of the synthesis, intracellular sorting, and function of glycoproteins using a mammalian cell mutant with reversible glycosylation defects.” Methods in Cell Biology 32: 57-84.
Kurzchalia, T. V. and R. G. Parton (1999). “Membrane microdomains and caveolae.” Current Opinion in Cell Biology 11(4): 424-31.
Kwan, C. P., G. Venkataraman, Z. Shriver, R. Raman, D. Liu, Y. Qi, L. Varticovski, R. Sasisekharan (2001). “Probing fibroblast growth factor dimerization and role of heparin-like glycosaminoglycans in modulating dimerization and signaling.” Journal of Biological Chemistry 276(26): 23421-9.
Li, M., J. Luo, C. L. Brooks, W. Gu (2002). “Acetylation of p53 inhibits its ubiquitination by Mdm2.” Journal of Biological Chemistry 277(52): 50607-11.
Li, W. and G. Keller (2000). “VEGF nuclear accumulation correlates with phenotypical changes in endothelial cells.” Journal of Cell Science 113 (Pt 9): 1525-34.
Liang, Y., M. Haring, P. J. Roughley, R. K. Margolis, R. U. Margolis (1997). “Glypican and biglycan in the nuclei of neurons and glioma cells: presence of functional nuclear localization signals and dynamic changes in glypican during the cell cycle.” Journal of Cell Biology 139(4): 851-64.
Lin, S. Y., K. Makino, W. Xia, A. Matin, Y. Wen, K. Y. Kwong, L. Bourguignon, M. C. Hung (2001). “Nuclear localization of EGF receptor and its potential new role as a transcription factor.” Nature Cell Biology 3(9): 802-8.
Longley, R. L., A. Woods, A. Fleetwood, G. J. Cowling, J. T. Gallagher, J. R. Couchman (1999). “Control of morphology, cytoskeleton and migration by syndecan-4.” Journal of Cell Science 112 (Pt 20): 3421-31.
Lu, Z., D. Liu, A. Hornia, W. Devonish, M. Pagano, D. A. Foster (1998). “Activation of protein kinase C triggers its ubiquitination and degradation.” Molecular and Cellular Biology 18(2): 839-45.
Luo, J., F. Su, D. Chen, A. Shiloh, W. Gu (2000). “Deacetylation of p53 modulates its effect on cell growth and apoptosis.” Nature 408(6810): 377-81.
Maher, P. A. (1996). “Nuclear translocation of fibroblast growth factor (FGF) receptors in response to FGF-2.” Journal of Cell Biology 134(2): 529-536.
Marmorstein, R. and S. Y. Roth (2001). “Histone acetyltransferases: function, structure, and catalysis.” Current Opinion in Genetics and Development 11(2): 155-61.
Marwick, J. A., Kirkham, P. A., Stevenson, C. S., Danahay, H., Giddings, J., Butler, K., Donaldson, K., Macnee, W. and Ralunan, I. (2004) Cigarette smoke alters chromatin remodeling and induces proinflammatory genes in rat lungs, Am J Respir Cell Mol Biol 31, 633-42
Martiny-Baron, G., M. G. Kazanietz, H. Mischak, P. M. Blumberg, G. Kochs, H. Hug, D. Marme, C. Schachtele (1993). “Selective inhibition of protein kinase C isozymes by the indolocarbazole Go 6976.” Journal of Biological Chemistry 268(13): 9194-7.
Miyamoto, S., B. Z. Katz, R. M. Lafrenie, K. M. Yamada (1998). “Fibronectin and integrins in cell adhesion, signaling, and morphogenesis.” Annals of the New York Academy of Sciences 857: 119-29.
Moscatelli, D. (1987). “High and low affinity binding sites for basic fibroblast growth factor on cultured cells: absence of a role for low affinity binding in the stimulation of plasminogen activator production by bovine capillary endothelial cells.” Journal of Cellular Physiology 131(1): 123-30.
Murakami J, T. Nishida, T. Otori (1992). “Coordinated appearance of b1 integrins and fibronectin during corneal wound healing.” Journal of Laboratory and Clinical Medicine 120: 86-93.
Murakami, M., A. Horowitz, S. Tang, J. A. Ware, M. Simons (2002). “Protein kinase C (PKC) delta regulates PKCalpha activity in a Syndecan-4-dependent manner.” Journal of Biological Chemistry 277(23): 20367-71.
Nabi, I. R. and P. U. Le (2003). “Caveolae/raft-dependent endocytosis.” Journal of Cell Biology 161(4): 673-7.
Nakanishi, Y., K. Kihara, K. Mizuno, Y. Masamune, Y. Yoshitake, K. Nishikawa (1992). “Direct effect of basic fibroblast growth factor on gene transcription in a cell-free system.” Proceedings of the National Academy of Sciences USA 89(12): 5216-20.
Neufeld, G., T. Cohen, S. Gengrinovitch, Z. Poltorak (1999). “Vascular endothelial growth factor (VEGF) and its receptors.” FASEB Journal 13(1): 9-22.
Newton, A. C. (1995). “Protein kinase C: structure, function, and regulation.” Journal of Biological Chemistry 270(48): 28495-28498.
Nichols, B. J. and J. Lippincott-Schwartz (2001). “Endocytosis without clathrin coats.” Trends in Cell Biology 11(10): 406-12.
Nishizuka, Y. (1995). “Protein kinase C and lipid signalling for sustained cellular responses.” FASEB Journal 9: 484-496.
Nugent, M. A. and E. R. Edelman (1992). “Kinetics of basic fibroblast growth factor binding to its receptor and heparan sulfate proteoglycan: a mechanism for cooperactivity.” Biochemistry 31(37): 8876-83.
Nugent, M. A. and R. V. Iozzo (2000). “Fibroblast growth factor-2.” International Journal of Biochemistry and Cell Biology 32(2): 115-20.
Oh, E. S., A. Woods, J. R. Couchman (1997a). “Multimerization of the cytoplasmic domain of syndecan-4 is required for its ability to activate protein kinase C.” Journal of Biological Chemistry 272(18): 11805-11.
Oh, E. S., A. Woods, J. R. Couchman (1997b). “Syndecan-4 proteoglycan regulates the distribution and activity of protein kinase C.” Journal of Biological Chemistry 272(13): 8133-6.
Oh, E. S., A. Woods, S. T. Lim, A. W. Theibert, J. R. Couchman (1998). “Syndecan-4 proteoglycan cytoplasmic domain and phosphatidylinositol 4,5-bisphosphate coordinately regulate protein kinase C activity.” Journal of Biological Chemistry 273(17): 10624-9.
Okada-Ban, M., J. P. Thiery, J. Jouanneau (2000). “Fibroblast growth factor-2.” International Journal of Biochemistry & Cell Biology 32(3): 263-7.
Orlandi, P. A. and P. H. Fishman (1998). “Filipin-dependent inhibition of cholera toxin: evidence for toxin internalization and activation through caveolae-like domains.” Journal of Cell Biology 141(4): 905-15.
Park, P. W., O. Reizes, M. Bernfield (2000). “Cell surface heparan sulfate proteoglycans: selective regulators of ligand-receptor encounters.” Journal of Biological Chemistry 275(39): 29923-29926.
Patel, M., M. Yanagishita, G. Roderiquez, D. C. Bou-Habib, T. Oravecz, V. C. Haskall, M. A. Norcross (1993). “Cell-surface heparan sulfate proteoglycan mediates HIV-1 infection of T-cell lines.” AIDS Research and Human Retroviruses 9(2): 167-74.
Patrie, K. M., M. J. Botelho, K. Franklin, I. M. Chiu (1999). “Site-directed mutagenesis and molecular modeling identify a crucial amino acid in specifying the heparin affinity of FGF-1.” Biochemistry 38(29): 9264-72.
Pazin, M. J. and J. T. Kadonaga (1997). “What's up and down with histone deacetylation and transcription?” Cell 89(3): 325-8.
Pytowski, B., T. W. Judge, T. E. McGraw (1995). “An internalization motif is created in the cytoplasmic domain of the transferrin receptor by substitution of a tyrosine at the first position of a predicted tight turn.” Journal of Biological Chemistry 270(16): 9067-73.
Quarto, N., F. Amalric (1994). “Heparan sulfate proteoglycans as transducers of FGF-2 signalling.” Journal of Cell Science 107: 3201-3212.
Rahman, I., Marwick, J. and Kirkham, P. (2004) Redox modulation of chromatin remodeling: impact on histone acetylation and deacetylation, NF-kappaB and pro inflammatory gene expression, Biochem Pharmacol 68, 1255-67.
Ranganathan, R., E. M. Ross (1997). “PDZ domain proteins: scaffolds for signalling complexes.” Current Biology 7: R770-R773.
Rapraeger, A. and M. Bernfield (1985). “Cell surface proteoglycan of mammary epithelial cells. Protease releases a heparan sulfate-rich ectodomain from a putative membrane-anchored domain.” Journal of Biological Chemistry 260(7): 4103-9.
Rapraeger, A. and C. Yeaman (1989). “A quantitative solid-phase assay for identifying radiolabeled glycosaminoglycans in crude cell extracts.” Annals of Biochemistry 179(2): 361-5.
Rapraeger, A. C. (2000). “Syndecan-regulated receptor signaling.” Journal of Cell Biology 149(5): 995-8.
Rapraeger, A. C. and V. L. Ott (1998). “Molecular interactions of the syndecan core proteins.” Current Opinion in Cell Biology 10(5): 620-8.
Reilly, J. F. and P. A. Maher (2001). “Importin beta-mediated nuclear import of fibroblast growth factor receptor: role in cell proliferation.” Journal of Cell Biology 152(6): 1307-12.
Ricci, V., A. Galmiche, A. Doye, V. Necchi, E. Solcia, P. Boquet (2000). “High cell sensitivity to Helicobacter pylori VacA toxin depends on a GPI-anchored protein and is not blocked by inhibition of the clathrin-mediated pathway of endocytosis.” Molecular Biology of the Cell 11(11): 3897-909.
Richardson, T. P., V. Trinkaus-Randall, M. A. Nugent (1999). “Regulation of basic fibroblast growth factor binding and activity by cell density and heparan sulfate.” Journal of Biological Chemistry 274(19): 13534-13540.
Richardson, T. P., V. Trinkaus-Randall, M. A. Nugent (2000). “Regulation of heparan sulfate proteoglycan expression and nuclear localization by fibronectin.” Journal of Cell Science 114: 1613-1623.
Robinson, C. J. and S. E. Stringer (2001). “The splice variants of vascular endothelial growth factor (VEGF) and their receptors.” Journal of Cell Science 114 (Pt 5): 853-65.
Rodal, S. K., G. Skretting, O. Garred, F. Vilhardt, B. van Deurs, K. Sandvig (1999). “Extraction of cholesterol with methyl-beta-cyclodextrin perturbs formation of clathrin-coated endocytic vesicles.” Molecular Biology of the Cell 10(4): 961-74.
Roden, L., S. Ananth, P. Campbell, T. Curenton, G. Ekborg, S. Manzanella, D. Pillion, E. Meezan (1992). “Heparin—an introduction.” Advances in Experimental Medicine and Biology 313: 1-20.
Roghani, M. and D. Moscatelli (1992). “Basic fibroblast growth factor is internalized through both receptor-mediated and heparan sulfate-mediated mechanisms.” Journal of Biological Chemistry 267(31): 22156-62.
Rothberg, K. G., J. E. Heuser, W. C. Donzell, Y. S. Ying, J. R. Glenney, R. G. Anderson (1992). “Caveolin, a protein component of caveolae membrane coats.” Cell 68(4): 673-82.
Rothberg, K. G., Y. S. Ying, B. A. Kamen, R. G. Anderson (1990). “Cholesterol controls the clustering of the glycophospholipid-anchored membrane receptor for 5-methyltetrahydrofolate.” Journal of Cell Biology 111 (6 Pt 2): 2931-8.
Rykova, V. I. and E. V. Grigorieva (1998). “Proteoglycan composition in cell nuclei of mouse hepatoma.” Biochemistry (Moscow) 63(11): 1271-6.
Salmivirta, M., K. Lidholt, U. Lindahl (1996). “Heparan sulfate: a piece of information.” FASEB Journal 10(11): 1270-9.
Schraw, W., Y. Li, M. S. McClain, F. G. van der Goot, T. L. Cover (2002). “Association of Helicobacter pylori vacuolating toxin (VacA) with lipid rafts.” Journal of Biological Chemistry 277(37): 34642-50.
Shriver, Z., D. Liu, R. Sasisekharan (2002). “Emerging views of heparan sulfate glycosaminoglycan structure/activity relationships modulating dynamic biological functions.” Trends in Cardiovascular Medicine 12(2): 71-7.
Simons, K. and D. Toomre (2000). “Lipid rafts and signal transduction.” Nature Review Molecular and Cell Biology 1(1): 31-9.
Skretting, G., M. L. Torgersen, B. van Deurs, K. Sandvig (1999). “Endocytic mechanisms responsible for uptake of GPI-linked diphtheria toxin receptor.” Journal of Cell Science 112 (Pt 22): 3899-909.
Song, B. H., G. C. Lee, M. S. Moon, Y. H. Cho, C. H. Lee (2001). “Human cytomegalovirus binding to heparan sulfate proteoglycans on the cell surface and/or entry stimulates the expression of human leukocyte antigen class I.” Journal of General Virology 82 (Pt 10): 2405-13.
Sperinde, G. and M. Nugent (1998). “Heparan sulfate proteoglycans control intracellular processing of bFGF in vascular smooth muscle cells.” Biochemistry 37(38): 13153-13164.
Sperinde G. and M. Nugent (2000). “Mechanisms of fibroblast growth factor 2 intracellular processing: a kinetic analysis of the role of heparan sulfate proteoglycans.” Biochemistry 39: 3788-3796.
Sterner, D. E. and S. L. Berger (2000). “Acetylation of histones and transcription-related factors.” Microbiology and Molecular Biology Reviews 64(2): 435-59.
Sugahara, K. and H. Kitagawa (2002). “Heparin and heparan sulfate biosynthesis.” IUBMB Life 54(4): 163-75.
Sugita, K., K. Koizumi, H. Yoshida (1992). “Morphological reversion of sis-transformed NIH3T3 cells by trichostatin A.” Cancer Research 52(1): 168-72.
Szebenyi, G., J. F. Fallon (1999). “Fibroblast growth factors as multifunctional signaling factors.” International Review of Cytology 185: 45-106.
Tkachenko, E. and M. Simons (2002). “Clustering induces redistribution of syndecan-core protein into raft membrane domains.” Journal of Biological Chemistry 277(22): 19946-51.
Tumova, S., A. Woods, J. R. Couchman (2000). “Heparan sulfate proteoglycans on the cell surface: versatile coordinators of cellular functions.” International Journal of Biochemistry & Cell Biology 32(3): 269-88.
Turnbull, J., A. Powell, S. Guimond (2001). “Heparan sulfate: decoding a dynamic multifunctional cell regulator.” Trends in Cell Biology 11(2): 75-82.
Ushio-Fukai, M., L. Hilenski, N. Santanam, P. L. Becker, Y. Ma, K. K. Griendling, R. W. Alexander (2001). “Cholesterol depletion inhibits epidermal growth factor receptor transactivation by angiotensin II in vascular smooth muscle cells: role of cholesterol-rich microdomains and focal adhesions in angiotensin II signaling.” Journal of Biological Chemistry 276(51): 48269-75.
Volk, R., J. J. Schwartz, J. Li, R. D. Rosenberg, M. Simons (1999). “The role of syndecan cytoplasmic domain in basic fibroblast growth factor-dependent signal transduction.” Journal of Biological Chemistry 274(34): 24417-24424.
Watson, K., N. J. Gooderham, D. S. Davies, R. J. Edwards (1999). “Nucleosomes bind to cell surface proteoglycans.” Journal of Biological Chemistry 274(31): 21707-13.
Wells, A. and U. Marti (2002). “Signalling shortcuts: cell-surface receptors in the nucleus?” Nature Review Molecular & Cell Biology 3(9): 697-702.
Whitcher, J. P., M. Srinivasan, M. P. Upadhyay (2001). “Corneal blindness: a global perspective.” Bulletin of the World Health Organization 79(3): 214-21.
Wijelath, E. S., J. Murray, S. Rahman, Y. Patel, A. Ishida, K. Strand, S. Aziz, C.
Cardona, W. P. Hammond, G. F. Savidge, S. Rafii, M. Sobel (2002). “Novel vascular endothelial growth factor binding domains of fibronectin enhance vascular endothelial growth factor biological activity.” Circulation Research 91(1): 25-31.
Wilkinson, S. E., P. J. Parker, J. S, Nixon (1993). “Isoelizyme specificity of bisindolylmaleimides, selective inhibitors of protein kinase C.” Biochemical Journal 294 (Pt 2): 335-7.
Williams, K. J. and I. V. Fuki (1997). “Cell-surface heparan sulfate proteoglycans: dynamic molecules mediating ligand catabolism.” Current Opinion in Lipidology 8(5): 253-62.
Woods, A. and J. R. Couchman (1994). “Syndecan 4 heparan sulfate proteoglycan is a selectively enriched and widespread focal adhesion component.” Molecular Biology of the Cell 5(2): 183-92.
Woods, A., R. L. Longley, S. Tumova, J. R. Couchman (2000). “Syndecan-4 binding to the high affinity heparin-binding domain of fibronectin drives focal adhesion formation in fibroblasts.” Archives of Biochemistry and Biophysics 374(1): 66-72.
Yamada, K. M. (2000). “Fibronectin peptides in cell migration and wound repair.” Journal of Clinical Investigation 105(11): 1507-9.
Yoshida, M., M. Kijima, M. Akita, T. Beppu (1990). “Potent and specific inhibition of mammalian histone deacetylase both in vivo and in vitro by trichostatin A.” Journal of Biological Chemistry 265(28): 17174-9.
Zieske, J. D., S. C. Higashijima, S. J. Spurr-Michaud, I. K. Gipson (1987). “Biosynthetic responses of the rabbit cornea to a keratectomy wound.” Investigative Opthalmology & Visual Science 28(10): 1668-77.

We claim:

1. A method for inhibiting a histone acetyltransferase comprising contacting a histone acetyltransferase, or a substrate of a histone acetyltransferase, with a glycosaminoglycan.

2. A method for treating a disorder associated with hyperacetylation comprising administering to a patient having the disorder an effective amount of a pharmaceutical composition containing as its active agent a glycosaminoglycan oligosaccharide to inhibit a histone acetyltransferase.

3. The method of claim 1 or 2, wherein the glycosaminoglycan is heparin or heparan sulfate (HS).

4. The method of claim 3, wherein the heparin or heparan sulfate oligosaccharide is an oligosaccharide that does not contain O-sulfation on the 2 position of the uronic acid residues.

5. The method of claim 3, wherein the heparin or heparan sulfate oligosaccharide is an oligosaccharide that does not contain O-sulfation on the 6 position of glucosamine residues.

6. The method of claim 2, wherein the active agent is a heparan sulfate proteoglycan ectodomain.

7. The method of claim 2, wherein the active agent is a heparan sulfate proteoglycan ectodomain isolated from corneal stromal fibroblasts or from pulmonary fibroblasts.

8. The method of claim 1 or 2, wherein the glycosaminoglycan is selected from the group consisting of chrondroitin sulfate (CS), heparin (H), heparan sulfate (HS), hyaluronan (HA) and keratan sulfate (KS).

9. The method of claims 1 or 2, wherein the glycosaminoglycan oligosaccharide is an oligosaccharide of at least 5 sugar units in length.

10. The method of claim 9, wherein the glycosaminoglycan oligosaccharide is an oligosaccharide of at least 6 sugar units in length

11. The method of claim 9, wherein the glycosaminoglycan oligosaccharide is an oligosaccharide of 8-18 sugar units in length.

12. The method of claim 9, wherein the glycosaminoglycan oligosaccharide is an oligosaccharide of 8-12 sugar units in length.

13. The method of claim 2, wherein the active agent is a glycosaminoglycan that has been chemically or enzymatically modified.

14. The method of claim 2, wherein one further administers an agent that enhances nuclear uptake of the glycosaminoglycan.

15. The method of claim 14, wherein the agent that enhances nuclear uptake of the glycosaminoglycan is a polyaminoester.

16. The method of claim 2, wherein the disorder associated with hyperacetylation is a chronic obstructive pulmonary disease.

17. The method of claim 16, wherein the chronic obstructive pulmonary disease is emphysema or asthma.

18. The method of claim 17, wherein the chronic obstructive pulmonary disease is late stage asthma.

19. The method of claim 2, wherein the disorder associated with hyperacetylation is cancer, cardiovascular disease, or proliferative eye disease.

20. The method of claim 19, wherein the cardiovascular disease is not restenosis.
